(19) United States
(12) Patent Application Publication
(10) Pub. No.: US 2022/0267289 A1
(43) Pub. Date: Aug. 25, 2022

(54) SELECTIVE BCRP/ABCG2 TRANSPORTER INHIBITORS AS AGENTS TO ABOLISH RESISTANCE TO ANTI-CANCER AGENTS

(71) Applicants: UNIVERSITE GRENOBLE ALPES, Saint Martin d'Heres (FR); CNRS - CENTRE NATIONAL DE LA RECHERCHE SCIENTIFIQUE, Paris (FR); UNIVERSITE CLAUDE BERNARD LYON 1, Villeurbanne (FR)

(72) Inventors: Ahcène BOUMENDJEL, Meylan (FR); Pierre FALSON, Annonay (FR); Alexis MORENO, Lyon (FR); Basile PERES, Villard Bonnot (FR); Emile ROUSSEL, Grenoble (FR)

(73) Assignees: UNIVERSITE GRENOBLE ALPES, Saint Martin d'Heres (FR); CNRS - CENTRE NATIONAL DE LA RECHERCHE SCIENTIFIQUE, Paris (FR); UNIVERSITE CLAUDE BERNARD LYON 1, Villeurbanne (FR)

(21) Appl. No.: 17/629,966

(22) PCT Filed: Jun. 1, 2020
(86) PCT No.: PCT/IB2020/055168

§ 371 Date: Jan. 25, 2022

(30) Foreign Application Priority Data
Jul. 25, 2019 (FR) 1908485

(51) Int. Cl. C07D 311/66

(52) U.S. Cl. CPC C07D 311/66

(57) ABSTRACT

A compound of formula (I):

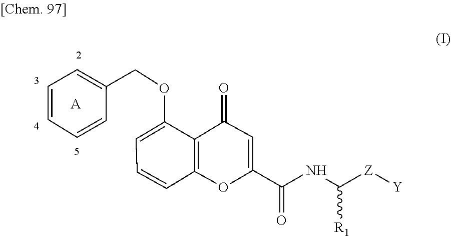

or pharmaceutically acceptable enantiomer, salt or solvate thereof, or a mixture thereof, the ring A, and the substituents Z, Y and R₁ being as defined herein.

The present invention relates to new compounds, their production process and their use as BCRP/ABCG2 inhibitors.

These new organic compounds all belong to the same chemical family derived from a natural compound: chromone.

Their main aim is to selectively inhibit the BCRP protein (Breast Cancer Resistance Protein), also known as ABCG2, which is involved in the phenomena of multidrug resistance of tumours to anticancer agents. BCRP acts as an efflux pump and is overexpressed in tumour cell membranes.

These compounds act as selective, non-cytotoxic inhibitors of BCRP/ABCG2.

Finally, these compounds have a better inhibitory activity and a better selectivity and are less cytotoxic than Ko143, the reference compound, while having a much simpler organic synthesis. In the clinic, these compounds can be used as adjuvants in combination with anti-cancer drugs in order to potentiate the effect of the latter and counteract the resistance of tumours to treatments aimed at eradicating them.

According to the WHO, cancer is the second leading cause of death in the world, causing almost 9.6 million deaths per year. The annual cost of treatments for this disease was estimated at 1160 billion dollars in 2010. Currently there are three main strategies for treating cancer depending on its stage and type: 1) surgery, 2) radiotherapy and 3) chemotherapy. It should be noted that these strategies can be used in addition to each other to complement the overall effectiveness of the treatment.

However, in chemotherapy, repeated use of the same anti-cancer agent causes the tumour cell to react in a protective and defensive manner against it. Unfortunately this will also induce an insensitivity of the tumour to a wide range of anti-cancer drugs, making chemotherapy ineffective and increasing the harm to the patient. One of the defences set up by tumour cells includes the overexpression in the cell membrane of transmembrane proteins called ATP-Binding Cassette (ABC) transporters. (Borst, P.; Elferink, R. O. Mammalian ABC Transporters in Health and Disease. Annu. Rev. Biochem. 2002, 71 (1), 537-592. Linton, K. J. Structure and Function of ABC Transporters. Physiology 2007, 22 (2), 122-130. Sharom, F. J. ABC Multidrug Transporters: Structure, Function and Role in Chemoresistance. Pharmacogenomics 2008, 9 (1), 105-127.)

Three of the 48 transmembrane proteins in this superfamily have been clearly identified as having a major role in chemotherapy failure: P-gp/ABCB1, MRP1/ABCC1, and BCRP/ABCG2 (Leslie, E. M.; Deeley, R. G.; Cole, S. P. C. Multidrug Resistance Proteins: Role of P-Glycoprotein, MRP1, MRP2, and BCRP (ABCG2) in Tissue Defense. Toxicol. Appl. Pharmacol. 2005, 204 (3), 216-237. Eckford, P. D. W.; Sharom, F. J. ABC Efflux Pump-Based Resistance to Chemotherapy Drugs. Chem. Rev. 2009, 109 (7), 2989-3011.)

They allow the transport of a wide class of chemical compounds and are naturally present in most cell membranes and physiological barriers in our body. Therefore, their physiological role in a healthy person is to defend and protect organelles and tissues from exogenous and/or xenobiotic agents. However, it has been shown that these ABC transporters drastically alter the absorption, distribution, metabolisation and elimination of active ingredients. (Sharom, F. J. ABC Multidrug Transporters: Structure, Function and Role in Chemoresistance. Pharmacogenomics 2008, 9 (1), 105-127.)

Together, these functions make these three transporters valid, novel and prime therapeutic targets in the search for a viable therapeutic solution to suppress chemoresistance problems. (Bugde, P.; Biswas, R.; Merien, F.; Lu, J.; Liu, D.-X.; Chen, M.; Zhou, S.; Li, Y. The Therapeutic Potential of Targeting ABC Transporters to Combat Multi-Drug Resistance. Expert Opin. Ther. Targets 2017, 21 (5), 511-530.)

Ko143 is a polycyclic organic molecule, containing 3 asymmetric centres, used today as a reference inhibitor on BCRP in research. However, its optimised 5-step synthesis is tedious with an overall yield of 5% and the control of the 3 asymmetric centres remains a limiting parameter (Li, Y.; Hayman, E.; Plesescu, M.; Prakash, S. R. Synthesis of Potent BCRP Inhibitor-Ko143. Tetrahedron Lett. 2008, 49 (9), 1480-1483.)

Despite good activity (50% inhibitory concentration, IC50=0.09 μM±0.01), this compound has a relatively low solubility affecting its bioavailability. It has been shown in clinical studies that Ko143 is rapidly metabolised (60 min.) via hydrolysis of its tertiary butyl ester, producing an inactive metabolite. (Liu, K.; Zhu, J.; Huang, Y.; Li, C.; Lu, J.; Sachar, M.; Li, S.; Ma, X. Metabolism of K0143, an ABCG2 inhibitor. Drug Metab. Pharmacokinet. 2017, 32 (4), 193-200.) As a result, clinical studies were stopped. Finally, this compound presented as selective for BCRP at the beginning, was eventually found to be non-selective for ABCG2 (Weidner, L. D.; Zoghbi, S. S.; Lu, S.; Shukla, S.; Ambudkar, S. V.; Pike, V. W.; Mulder, J.; Gottesman, M. M.; Innis, R. B.; Hall, M. D. The Inhibitor Ko143 Is Not Specific for ABCG2. J. Pharmacol. Exp. Ther. 2015, 354 (3), 384-393. Allen, J. D.; van Loevezijn, A.; Lakhai, J. M.; van der Valk, M.; van Tellingen, O.; Reid, G.; Schellens, J. H. M.; Koomen, G.-J.; Schinkel, A. H. Potent and Specific Inhibition of the Breast Cancer Resistance Protein Multidrug Transporter in Vitro and in Mouse Intestine by a Novel Analogue of Fumitremorgin C. Mol. Cancer Ther. 2002, 1 (6), 417.) The present applicants have developed in the past an inhibitor, called MBL-II-141, which is selective, nontoxic and with good activity in preclinical models. (Honorat, M.; Guitton, J.; Gauthier, C.; Bouard, C.; Lecerf-Schmidt, F.; Peres, B.; Terreux, R.; Gervot, H.; Rioufol, C.; Boumendjel, A.; et al. MBL-II-141, a Chromone Derivative, Enhances Irinotecan (CPT-11) Anticancer Efficiency in ABCG2-Positive Xenografts. Oncotarget 2014, 5 (23), 11957-11970. Henin, E.; Honorat, M.; Guitton, J.; Di Pietro, A.; Payen, L.; Tod, M. Pharmacokinetic Interactions in Mice between Irinotecan and MBL-II-141, an ABCG2 Inhibitor: Irinotecan MBLI-II-141 Interaction. Biopharm. Drug Dispos. 2017. Valdameri, G.; Genoux-Bastide, E.; Peres, B.; Gauthier, C.; Guitton, J.; Terreux, R.; Winnischofer, S. M. B.; Rocha, M. E. M.; Boumendjel, A.; Di Pietro, A. Substituted Chromones as Highly Potent Nontoxic Inhibitors, Specific for the Breast Cancer Resistance Protein. J. Med. Chem. 2012, 55 (2), 966-970. Lecerf-Schmidt, F.; Peres, B.; Valdameri, G.; Gauthier, C.; Winter, E.; Payen, L.; Di Pietro, A.; Boumendjel, A. ABCG2: Recent Discovery of Potent and Highly Selective Inhibitors. Future Med. Chem. 2013, 5 (9), 1037-1045. Winter, E.; Lecerf-Schmidt, F.; Gozzi, G.; Peres, B.; Lightbody, M.; Gauthier, C.; Ozvegy-Laczka, C.; Szakacs, G.; Sarkadi, B.; Creczynski-Pasa, T. B.; et al. Structure-Activity Relationships of Chromone Derivatives toward the Mechanism of Interaction with and Inhibition of Breast Cancer Resistance Protein ABCG2. J. Med. Chem. 2013, 56 (24), 9849-9860. Pires, A. do R. A.; Lecerf-Schmidt, F.; Guragossian, N.; Pazinato, J.; Gozzi, G. J.; Winter, E.; Valdameri, G.; Veale, A.; Boumendjel, A.; Di Pietro, A.; et al. New, Highly Potent and Non-Toxic, Chromone Inhibitors of the Human Breast Cancer Resistance Protein ABCG2. Eur. J. Med. Chem. 2016, 122, 291-301.). The compounds used as synthetic intermediates in this latest publication have now been found to be inhibitors of the multi-drug resistance protein of breast cancer (Breast Cancer Resistance Protein BCRP/ABCG2).

Chemoresistance problems are on the rise due to a lack of rapid renewal of anti-cancer agents on the market. In order to counter the lack of new anti-cancer agents, positioning oneself at the source of the resistance problem is a viable and economical solution. Indeed, suppressing the ability of tumour cells to protect and defend themselves preserves the efficacy of current anti-cancer drugs and limits their active dose, which in turn limits their adverse effects and the overall cost of chemotherapy for the patient and society. Moreover, it would be interesting to find new compounds that are even more effective than MBL-II-141 and Ko143.

The present invention relates to compounds of formula (I):

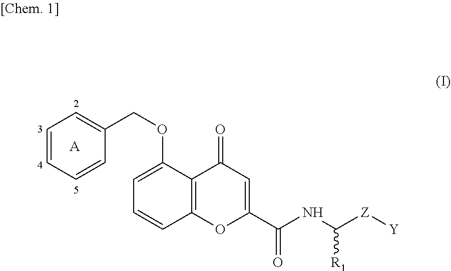

or pharmaceutically acceptable enantiomer, salt or solvate thereof, or a mixture thereof,
in which:
the ring A is unsubstituted or substituted in the 2, 3, 4, 5 position by one or two of F; Cl; Br; I; OR, with R=Me, Et, Pr, i-Pr, n-Bu; O—CH₂—(O—CH₂CH₂)ₙ—O—CH₃, with n=3, 4, 5, 6,
Z is

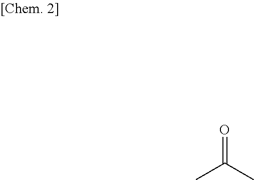

### or —CH₂—,

Y═—OH; —OMe; —OEt; —OPr; —NH₂; —NHMe; —N(Me)₂; —N(Me) OCH₃; —NH—(CH₂)₂-(3-indolyl); —NH(CH₂)₂-3-((5-hydroxy)indolyl); —NH—CH(R₃)—COR₂, with R₂ selected from:
   —OH; —OMe;-OEt; —OPr; —NH₂; —NHMe; —N(Me)₂; —N (Me) OCH₃; 3-(5-methoxy) indolyl; —NH—(CH₂)₂-(3-indolyl); —NH(CH₂)₂-3-((5-hydroxy) indolyl) —NH(CH₂)₂-3-((5-methoxy) indolyl)

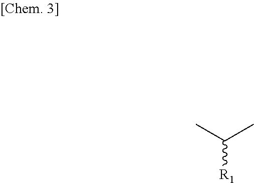

in formula (I) and R₃ of the substituent —NH—CH(R₃)—COR₂ of Y are independently selected from: H or

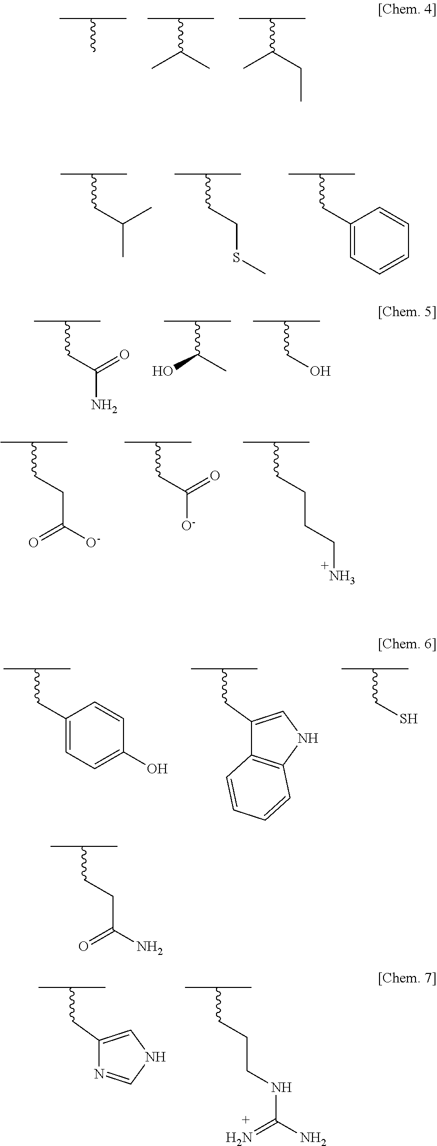

with the exception of compounds with simultaneous Br in the 4-position of ring A, R₁═CH(CH₃)₂ or CH₂CH(CH₃)₂ or CH(CH₃)CH₂CH₃, Z=

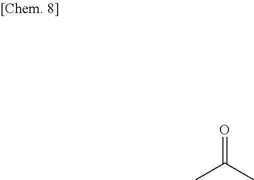

### and Y═—OH or —OMe.

In particular, the ring A may be substituted in position 2, 3, 4, 5 by one or two Br and Y═—OH; —OMe; —NH—(CH₂)₂-(3-indolyl); —NH(CH₂)₂-3-((5-hydroxy) indolyl); —NH—CH(R₃)—COR₂, R₁, R₂ and R₃ being as defined above.

In a particular embodiment, Y is —NH—(CH₂)₂-(3-indolyl) or —NH(CH₂)₂-3-((5-hydroxy) indolyl) provided that:

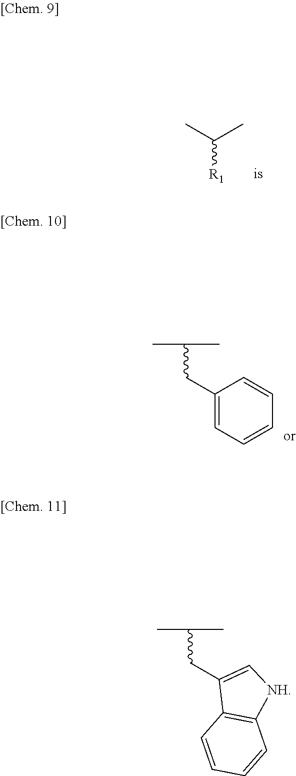

In particular, the compounds according to the invention may be selected from:
methyl (5-((2-bromobenzyl)oxy)-4-oxo-4H-chromene-2-carbonylamino)-L-alloisoleucinate;
methyl (5-((2-bromobenzyl)oxy)-4-oxo-4H-chromene-2-carbonylamino)-L-leucinate;
methyl (5-((2-bromobenzyl)oxy)-4-oxo-4H-chromene-2-carbonylamino)-L-valinate;
methyl (5-((2-bromobenzyl)oxy)-4-oxo-4H-chromene-2-carbonylamino)-L-phenylalaninate;
methyl (5-((4-bromobenzyl)oxy)-4-oxo-4H-chromene-2-carbonylamino)-L-phenylalaninate;
methyl (5-((2-bromobenzyl)oxy)-4-oxo-4H-chromene-2-carbonylamino)-L-tryptophanate;
methyl (5-((4-bromobenzyl)oxy)-4-oxo-4H-chromene-2-carbonylamino)-L-tryptophanate;
methyl (5-((4-bromobenzyl)oxy)-4-oxo-4H-chromene-2-carbonylamino)-D-tryptophanate;
(5-((2-bromobenzyl)oxy)-4-oxo-4H-chromene-2-carbonylamino)-L-alloisoleucine;
(5-((2-bromobenzyl)oxy)-4-oxo-4H-chromene-2-carbonylamino)-L-phenylalanine;
(5-((4-bromobenzyl)oxy)-4-oxo-4H-chromene-2-carbonylamino)-L-phenylalanine;
(5-((4-bromobenzyl)oxy)-4-oxo-4H-chromene-2-carbonylamino)-D-tryptophan;
methyl (5-((2-bromobenzyl)oxy)-4-oxo-4H-chromene-2-carbonylamino)-L-alloisoleucyl-L-valinate;
methyl (5-((2-bromobenzyl)oxy)-4-oxo-4H-chromene-2-carbonylamino)-L-alloisoleucyl-L-leucinate;
methyl (5-((4-bromobenzyl)oxy)-4-oxo-4H-chromene-2-carbonylamino)-L-alloisoleucyl-L-valinate;
methyl (5-((4-bromobenzyl)oxy)-4-oxo-4H-chromene-2-carbonylamino)-L-alloisoleucyl-L-leucinate;
(S)-5-((2-bromobenzyl)oxy)-N-(1-((2-(5-hydroxy-1H-indol-3-yl)ethyl)amino)-1-oxo-3-phenylpropan-2-yl)-4-oxo-4H-chromene-2-carboxamide;
(S)—N-(1-((2-(1H-indol-3-yl)ethyl)amino)-1-oxo-3-phenylpropan-2-yl)-5-((4-bromobenzyl)oxy)-4-oxo-4H-chromene-2-carboxamide; and
le (R)—N-(1-((2-(1H-indol-3-yl)éthyl)amino)-3-(1H-indol-3-yl)-1-oxopropan-2-yl)-5-((4-bromobenzyl)oxy)-4-oxo-4H-chromene-2-carboxamide.

The present invention also relates to a process for obtaining the compounds according to the invention, characterised by the fact that it comprises the steps
(a) an alkylating compound of the formula

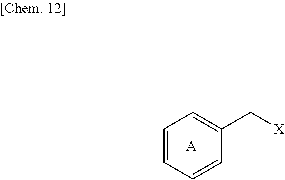

Wherein the ring A is as defined in claim 1, and X is a halogen selected from F, Cl, Br and I, is reacted on 2,6-dihydroxyacetophenone of the formula

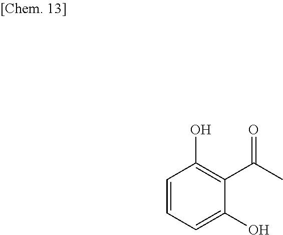

at the reflux temperature of acetone and in acetone to give the intermediate of formula

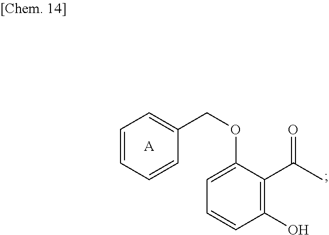

(b) the intermediate obtained in step (a) is reacted with diethyl oxalate of formula

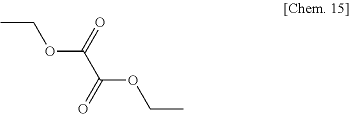

at a temperature of 0° C.-50° C. and in a mixture of tetrahydrofuran (THF)/ethanol (1:1) to give the intermediate of formula

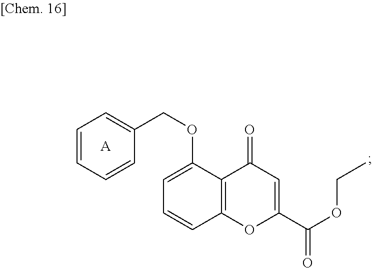

(c) the intermediate obtained in step (b) is reacted by a hydrolysis reaction of the ester function at a temperature of 50° C., in an acidic or basic medium, in a THF/ethanol/water solvent (3:1:1.5) in order to obtain the intermediate of formula

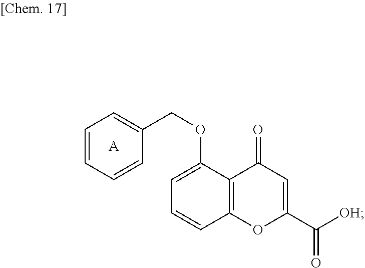

(d) the intermediate obtained in step (c) is reacted with a coupling compound of the formula

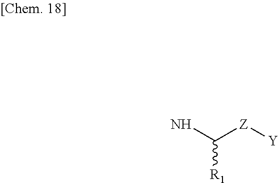

R₁, Z and Y being as defined in claim 1, at room temperature in anhydrous DMF to form an amide bond to give the compound of formula (I).

The present invention also relates to a compound of formula (I):

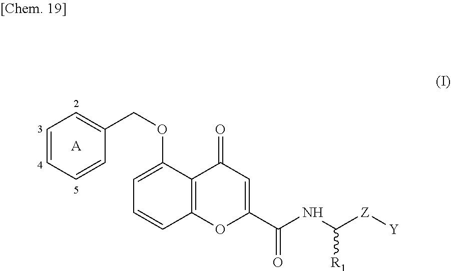

or pharmaceutically acceptable enantiomer, salt or solvate thereof, or a mixture thereof, in which:
the ring A is unsubstituted or substituted in the 2, 3, 4, 5 position by one or two of F; Cl; Br; I; OR, with R=Me, Et, Pr, i-Pr, n-Bu; O—CH₂—(O—CH₂CH₂)ₙ—O—CH₃, with n=3, 4, 5, 6,
Z is

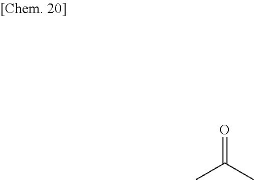

### or —CH₂—,

Y═—OH; —OMe;-OEt; —OPr; —NH₂; —NHMe; —N(Me)₂; —N(Me) OCH₃; —NH—(CH₂)₂-(3-indolyl); —NH(CH₂)₂-3-((5-hydroxy)indolyl); —NH—CH(R₃)—COR₂, with R₂ selected from:
   —OH; —OMe;-OEt; —OPr; —NH₂; —NHMe; —N(Me)₂; —N (Me) OCH₃; 3-(5-methoxy) indolyl; —NH—(CH₂)₂-(3-indolyl); —NH(CH₂)₂-3-((5-hydroxy) indolyl) —NH(CH₂)₂-3-((5-methoxy) indolyl)

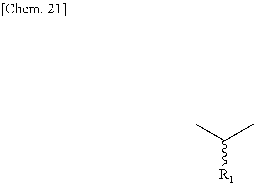

in formula (I) and R₃ of the substituent —NH—CH(R₃)—COR₂ of Y are independently selected from H or

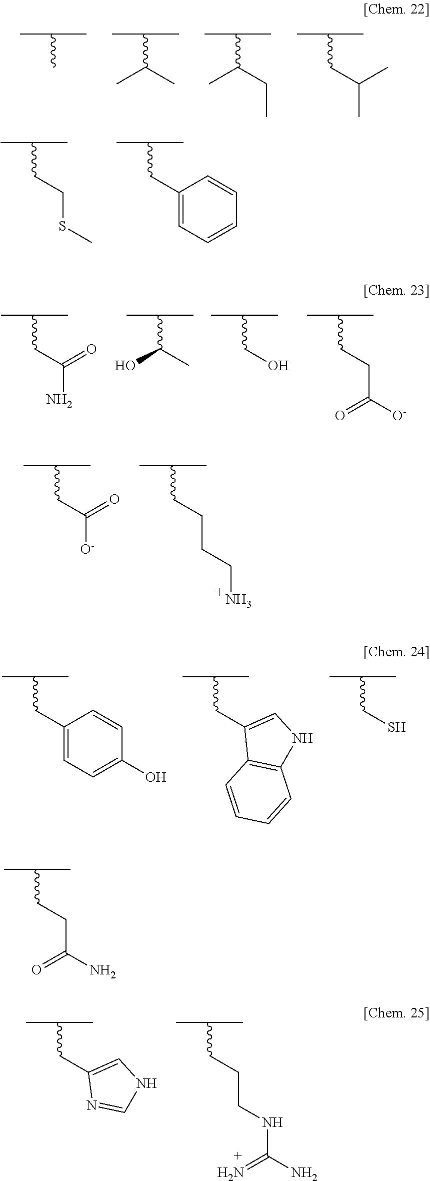

for its use in the inhibition of the multi-drug resistance protein of the breast cancer (Breast Cancer Resistance Protein BCRP/ABCG2).

Inhibition of the multi-drug resistance protein of the breast cancer (Breast Cancer Resistance Protein BCRP/ABCG2), in addition to its role in the treatment of breast cancer, may also allow for the re-sensitisation of tumour cells and the enhancement of the pharmacokinetics and efficacy of drugs that require passage of physiological membranes or barriers, such as the blood-brain or gastrointestinal barrier, in order to exhibit their therapeutic activities.

In particular, the ring A may be substituted in position 2, 3, 4, 5 by one or two Br and Y═—OH; —OMe; —NH—(CH₂)₂-(3-indolyl); —NH(CH₂)₂-3-((5-hydroxy) indolyl); —NH—CH(R₃)—COR₂, R₁, R₂ and R₃ being as defined above.

In a particular embodiment, Y is —NH—(CH₂)₂-(3-indolyl) or —NH(CH₂)₂-3-((5-hydroxy) indolyl) provided that:

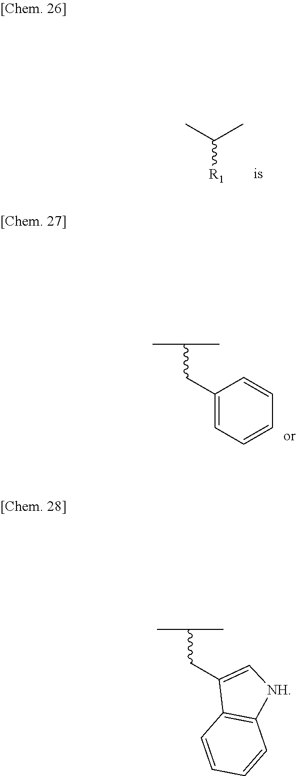

Compounds for use in the inhibition of the multi-drug resistance protein of the breast cancer (Breast Cancer Resistance Protein BCRP/ABCG2) according to the invention may be selected from:
methyl (5-((2-bromobenzyl)oxy)-4-oxo-4H-chromene-2-carbonylamino)-L-alloisoleucinate;
methyl (5-((4-bromobenzyl)oxy)-4-oxo-4H-chromene-2-carbonylamino)-L-alloisoleucinate;
methyl (5-((2-bromobenzyl)oxy)-4-oxo-4H-chromene-2-carbonylamino)-L-leucinate;
methyl (5-((2-bromobenzyl)oxy)-4-oxo-4H-chromene-2-carbonylamino)-L-valinate;
methyl (5-((2-bromobenzyl)oxy)-4-oxo-4H-chromene-2-carbonylamino)-L-phenylalaninate;
methyl (5-((4-bromobenzyl)oxy)-4-oxo-4H-chromene-2-carbonylamino)-L-phenylalaninate;
methyl (5-((2-bromobenzyl)oxy)-4-oxo-4H-chromene-2-carbonylamino)-L-tryptophanate;
methyl (5-((4-bromobenzyl)oxy)-4-oxo-4H-chromene-2-carbonylamino)-L-tryptophanate;
methyl (5-((4-bromobenzyl)oxy)-4-oxo-4H-chromene-2-carbonylamino)-D-tryptophanate;
(5-((2-bromobenzyl)oxy)-4-oxo-4H-chromene-2-carbonylamino)-L-alloisoleucine;
(5-((2-bromobenzyl)oxy)-4-oxo-4H-chromene-2-carbonylamino)-L-phenylalanine;
(5-((4-bromobenzyl)oxy)-4-oxo-4H-chromene-2-carbonylamino)-L-phenylalanine;
(5-((4-bromobenzyl)oxy)-4-oxo-4H-chromene-2-carbonylamino)-D-tryptophan;
methyl (5-((2-bromobenzyl)oxy)-4-oxo-4H-chromene-2-carbonylamino)-L-alloisoleucyl-L-valinate;
methyl (5-((2-bromobenzyl)oxy)-4-oxo-4H-chromene-2-carbonylamino)-L-alloisoleucyl-L-leucinate;
methyl (5-((4-bromobenzyl)oxy)-4-oxo-4H-chromene-2-carbonylamino)-L-alloisoleucyl-L-valinate;
methyl (5-((4-bromobenzyl)oxy)-4-oxo-4H-chromene-2-carbonylamino)-L-alloisoleucyl-L-leucinate;
(S)-5-((2-bromobenzyl)oxy)-N-(1-((2-(5-hydroxy-1H-indol-3-yl)ethyl)amino)-1-oxo-3-phenylpropan-2-yl)-4-oxo-4H-chromene-2-carboxamide;
(S)—N-(1-((2-(1H-indol-3-yl)ethyl)amino)-1-oxo-3-phenylpropan-2-yl)-5-((4-bromobenzyl)oxy)-4-oxo-4H-chromene-2-carboxamide, and
(R)—N-(1-((2-(1H-indol-3-yl)ethyl)amino)-3-(1H-indol-3-yl)-1-oxopropan-2-yl)-5-((4-bromobenzyl)oxy)-4-oxo-4H-chromene-2-carboxamide.

The present invention also relates to a pharmaceutical composition comprising:
at least one pharmaceutically acceptable active agent; and
at least one compound as defined above.

In particular, that the pharmaceutically acceptable active agent may be selected from anti-cancer agents, intestinal anti-inflammatory agents, hypocholesteremic agents, anti-dyslipidemic agents and kinase inhibitors.

For example, as an intestinal anti-inflammatory agent, Sulphasalazine may be used, as a hypocholesteremic agent, Atorvastatin may be used, as an anti-dyslipidemic agent, Rosuvastatin may be used, as an anti-cancer agent or kinase inhibitor, imatinib mesylate may be used.

These compounds, which can be distinguished by the presence of a natural amino acid or the enantiomer of a natural amino acid linked to a chromone unit, were synthesised in four steps from commercial 2,6-dihydroxyacetophenone 1 (Scheme 1) in an overall yield of between 13 and 41%. Intermediate 2 is accessed in three standard steps: alkylation, Kostaneki reaction and saponification, in 45% overall yield. The last step is carried out via a coupling reaction, for example a peptide coupling when a natural amino acid or the enantiomer of a natural amino acid is bound to the chromone unit, assisted by a coupling agent facilitating the reaction and limiting the number of equivalents of reactants.

The reagents are inexpensive and are typically used in the laboratory. Moreover, the reactions are not very energy consuming and the purification of the products by precipitation or recrystallisation limits the volume of organic solvents used and therefore the waste. Finally, the compounds were isolated in pure form and their structures were established and verified by various analytical techniques (NMR, mass spectrometry, X-ray diffraction). It should be noted that X-ray diffraction confirmed that there was no racemisation of the asymmetric centre of the commercial amino acid.

### Scheme 1: Synthesis Pathway of the BCRP Inhibitors of the Invention.

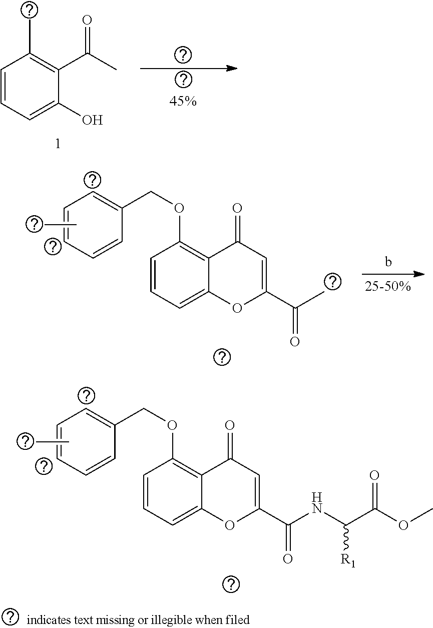

The following examples illustrate the present invention without limiting its scope.

In these Examples:

NMR spectra were recorded on a Bruker Avance-400 400 MHz instrument (400 MHz) or a Bruker Avance-500 500 MHz instrument (500 MHz).

Chemical shifts (5) were reported in ppm relative to Me₄Si used as internal standard.

Electrospray ionisation (ESI) mass spectra were acquired by the Analytical Department of the University of Grenoble on a Waters Xevo G2-S Q TOF instrument with a nano-spray input. The exact mass was given in m/z.

HPLC analyses were performed with an Agilent 1100 series system using a diode array detector and a C18 reverse phase column (Nucleosil C18, Macherey-Nagel, 5 mm particle size, 125 mm×3 mm) at 45° C., with a mobile phase consisting of A: water and 0.1% trifluoroacetic acid (TFA) and B: methanol (MeOH) and 0.1% TFA with an A:B gradient from 85:15 to 0:100 over 14 min, 1 mL/min, 10 μL injection, detection at 254 nm.

The melting points (m.p.) expressed in degrees Celsius (° C.) were obtained on a Buchi B540 melting point.

Thin layer chromatography (TLC) was performed on Merck F-254 silica gel plates (0.25 mm thick).

Unless otherwise stated, the reagents were obtained from commercial sources (Alpha Aesar, Sigma-Aldrich and TCI) and were used without further purification.

### EXAMPLES 1 TO 20

### Series 1

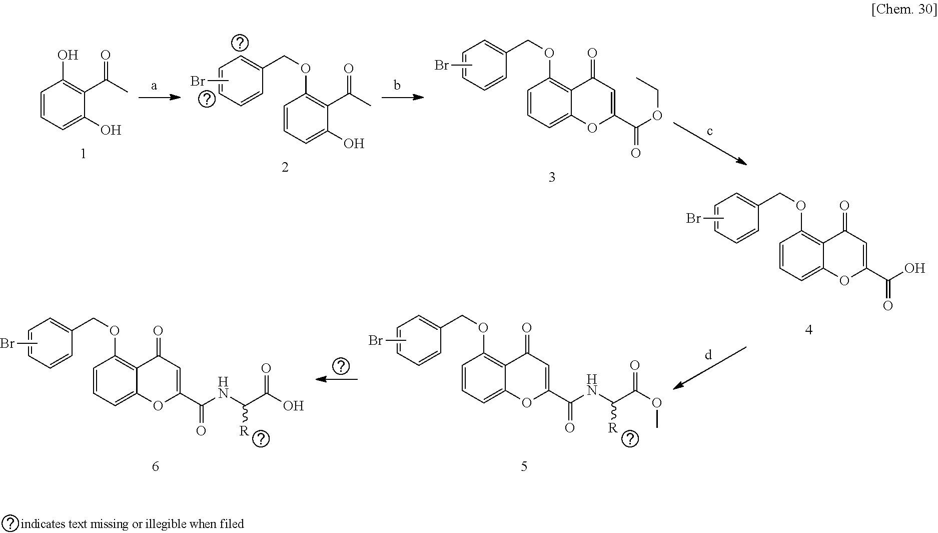

Note: In the following protocols, “molecule number+a” refers to when the bromine is in position 2 of the aromatic ring while “molecule number+b” refers to when the bromine is in position 4 of the aromatic ring.

### General Procedure A:

To a solution of 2,6-dihydroxyacetophenone 1 (1 equiv) in acetone (6 mL/mmol) was simultaneously added K₂CO₃ (3 equiv) and tetra-n-butylammonium bromide (0.4 equiv) previously homogenised together.

The solution was refluxed for 30-60 min before the dropwise addition of the corresponding bromobenzyl bromide (1 equiv) in acetone (15 mL/mmol).

The solution was then refluxed for 4-5 hours and monitored by TLC (ethyl acetate/cyclohexane 3:7). The solution was poured into water and extracted with ethyl acetate. The organic phases were collected and washed with water and brine before being dried over magnesium sulphate, filtered and evaporated under vacuum.

### General Procedure B:

To a solution of 2 (1 equiv) in anhydrous THF (10 mL/mmol) was added a solution of sodium ethanolate generated in situ from sodium (6 equiv) in anhydrous ethanol (15 mL/mmol) at 0° C. and under an inert atmosphere.

The solution was stirred for 30 min at room temperature and diethyl oxalate (4 equiv) was added dropwise to the solution. The solution was then heated to 50° C. until precipitation and then refluxed for 2 hours. The reaction was monitored by TLC (cyclohexane/ethyl acetate 1:1).

Then a few drops of concentrated hydrochloric acid (37%) were added to the solution until the precipitate formed became white.

The solution was refluxed for 1 hour before being cooled to room temperature. After concentration under vacuum, the solution was poured into water and extracted with ethyl acetate. The organic phases were collected and washed with water and brine before being dried over magnesium sulphate, filtered and evaporated under vacuum.

### General Operating Procedure C

To a solution of 3 (1 equiv) in THF (25 mL/mmol) and ethanol (8 mL/mmol) was added a solution of K₂CO₃ (1.3 equiv) in water (12 mL/mmol).

The solution was heated to 50° C. for 4 hours and monitored by TLC (ethyl acetate/cyclohexane 1:1). The solution was concentrated under vacuum and then poured into basic water (K₂CO₃ 20%) and washed with ethyl acetate. The basic aqueous phase was acidified with concentrated hydrochloric acid (37%) and extracted with ethyl acetate.

The organic phases were then collected and washed with water and brine before being dried over magnesium sulphate, filtered and evaporated.

### General Operating Procedure D:

To a solution of carboxylic acid derivative 4 (1 equiv) in anhydrous dimethylformamide (DMF) (20 mL/mmol) was added 2-(1H-benzotriazole-1-yl)-1,1,3,3-tetramethylaminium tetrafluoroborate (TBTU) (2 equiv). The solution was stirred for 30 minutes at room temperature.

Next, a solution of amino acid derivative (2 equiv) in DMF (10 mL/mmol) in the presence of N, N-diisopropylethylamine (DIEA) (4 equiv) was added carefully to the previous one. The reaction was stirred for 12 or 24 hours at room temperature and monitored by TLC (ethyl acetate/cyclohexane 1:1). The solution was concentrated under vacuum and then poured into acidified water (1M HCl) and extracted with ethyl acetate.

The organic phases were collected and washed with NaHCO₃ solution (20%), water and brine before being dried over magnesium sulphate, filtered and concentrated under vacuum.

### General Operating Mode E

To a solution of ester derivative 5 (1 equiv) in THF (25 mL/mmol) and methanol (10 mL/mmol) was added a solution of LiOH (1.5 equiv) in H₂O (10 mL/mmol). The reaction was stirred for 2 hours at room temperature and monitored by TLC (ethyl acetate/cyclohexane 1:1).

The solution was poured into basified water (20% NaHCO₃) and washed with ethyl acetate. The aqueous phase was acidified with concentrated hydrochloric acid (37%) and extracted with ethyl acetate.

The organic phases were then collected and washed with brine before being dried over magnesium sulphate, filtered and concentrated under vacuum.

### EXAMPLE 1

### Preparation of 1-(2-((2-bromobenzyl)oxy)-6-hydroxyphenyl)ethan-1-one (2a)

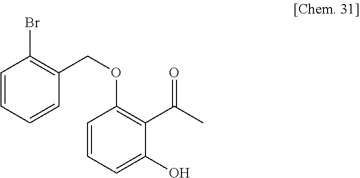 [SMILES: CC(=O)c1c(O)cccc1OCc1ccccc1Br]

The crude was prepared according to general procedure A starting from 1 (1.500 g, 9.86 mmol) and purified by recrystallisation in methanol or ethyl acetate to give 2a (2.583 g, 82%). C₁₅H₁₃BrO₃.

¹H NMR (400 MHz, DMSO) δ 11.69 (s, 1H), 7.71 (dd, J=8.0, 1.1 Hz, 1H), 7.61 (dd, J=7.6, 1.6 Hz, 1H), 7.46 (td, J=7.5, 1.2 Hz, 1H), 7.39-7.29 (m, 2H), 6.67 (dd, J=8.4, 0.5 Hz, 1H), 6.56 (dd, J=8.3, 0.7 Hz, 1H), 5.19 (s, 2H), 2.47 (s, 3H).

¹³C NMR (101 MHz, DMSO) δ 203.38, 159.60, 157.95, 135.31, 133.90, 132.72, 130.64, 130.41, 128.00, 123.04, 114.62, 109.83, 103.19, 70.02, 32.86

m. p.: 75.5-77.4° C.

MS (ESI) m/z 321 (⁷⁹Br), 323 (⁸¹Br) [M+H]⁺, 319 (⁷⁹Br), 321 (⁸¹Br) [M−H]⁺.

### EXAMPLE 2

### Preparation of 1-(2-((4-bromobenzyl)oxy)-6-hydroxyphenyl)ethan-1-one (2b)

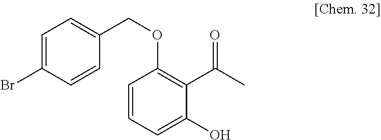 [SMILES: CC(=O)c1c(O)cccc1OCc1ccc(Br)cc1]

The crude was prepared according to general procedure A starting from 1 (1.500 g, 9.86 mmol) and purified by recrystallisation in ethyl acetate to give 2b (2.321, 73%). C₁₅H₁₃BrO₃.

¹H NMR (400 MHz, DMSO) δ 11.64 (s, 1H), 7.62-7.58 (m, 2H), 7.45-7.41 (m, 2H), 7.30 (t, J=8.3 Hz, 1H), 6.62 (dd, J=8.4, 0.5 Hz, 1H), 6.52 (dd, J=8.3, 0.7 Hz, 1H), 5.14 (s, 2H), 2.48 (s, 3H)

¹³C NMR (101 MHz, DMSO) δ 203.43, 159.52, 157.94, 135.96, 133.77, 131.41, 129.98, 121.15, 114.75, 109.63, 103.41, 69.30, 33.04

m. p.: 114.8-116.8° C.

MS (ESI) m/z 320 (⁷⁹Br), 322 (⁸¹Br) [M].

### EXAMPLE 3

### Preparation of ethyl 5-((2-bromobenzyl)oxy)-4-oxo-4H-chromene-2-carboxylate (3a)

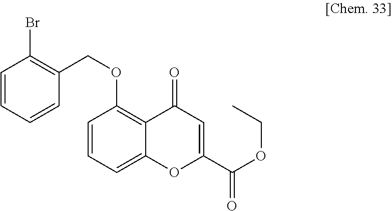 [SMILES: CCOC(=O)c1cc(=O)c2c(OCc3ccccc3Br)cccc2o1]

The crude was prepared according to general procedure B starting from 2a (2.583 g, 8.04 mmol) and purified by recrystallisation in methanol or ethyl acetate to give 3a (2.478, 76%). C₁₉H₁₅BrO₅.

¹H NMR (400 MHz, DMSO) δ 8.11 (dd, J=7.7, 1.4 Hz, 1H), 7.79 (t, J=8.4 Hz, 1H), 7.68 (dd, J=8.0, 1.0 Hz, 1H), 7.51 (td, J=7.6, 1.1 Hz, 1H), 7.33 (td, J=7.8, 1.7 Hz, 1H), 7.30-7.25 (m, 1H), 7.18-7.13 (m, 1H), 6.79 (s, 1H), 5.23 (s, 2H), 4.40 (q, J=7.1 Hz, 2H), 1.36 (t, J=7.1 Hz, 3H)

¹³C NMR (101 MHz, DMSO) δ 176.40, 172.49, 160.01, 158.25, 157.43, 150.24, 135.70, 135.44, 132.17, 129.57, 129.12, 127.85, 121.05, 115.43, 114.62, 110.78, 108.85, 69.72, 62.61, 13.87

m. p.: 155.3-157.1° C.

MS (ESI) m/z 403 (⁷⁹Br), 405 (⁸¹Br) [M−H]⁺.

### EXAMPLE 4

### Preparation of ethyl 5-((4-bromobenzyl)oxy)-4-oxo-4H-chromene-2-carboxylate (3b)

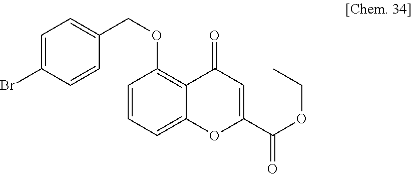 [SMILES: CCOC(=O)c1cc(=O)c2c(OCc3ccc(Br)cc3)cccc2o1]

The crude was prepared according to general procedure B starting from 2b (1.718 g, 5.35 mmol) and purified by recrystallisation in methanol or ethyl acetate to give 3b (1.516, 70%). C₁₉H₁₅BrO₅.

¹H NMR (400 MHz, DMSO) δ 7.74 (t, J=8.4 Hz, 1H), 7.64-7.56 (m, 4H), 7.25-7.21 (m, 1H), 7.13-7.08 (m, 1H), 6.77 (s, 1H), 5.24 (s, 2H), 4.38 (q, J=7.1 Hz, 2H), 1.34 (t, J=7.1 Hz, 3H).

¹³C NMR (101 MHz, DMSO) δ 176.39, 160.01, 157.70, 157.23, 150.19, 136.23, 135.29, 131.21, 128.90, 120.61, 115.43, 114.68, 110.54, 109.02, 69.22, 62.59, 13.87

m. p.: 148.0-149.4° C.

MS (ESI) m/z 402 (⁷⁹Br), 404 (⁸¹Br) [M]⁺.

### EXAMPLE 5

### Preparation of 5-((2-bromobenzyl)oxy)-4-oxo-4H-chromene-2-carboxylic Acid (4a)

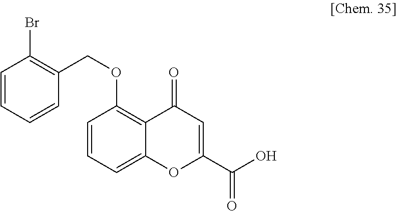 [SMILES: O=C(O)c1cc(=O)c2c(OCc3ccccc3Br)cccc2o1]

The crude was prepared according to general procedure C starting from 3a (1.000 g, 2.48 mmol) and purified by trituration in methanol and washed with diethyl ether to give 4a (0.777, 84%). The desired product can also be obtained crystalline by recrystallising in methanol. C₁₇H₁₁BrO₅.

¹H NMR (400 MHz, DMSO) δ 8.14 (dd, J=7.7, 1.1 Hz, 1H), 7.77 (t, J=8.4 Hz, 1H), 7.68 (dd, J=8.0, 0.9 Hz, 1H), 7.51 (td, J=7.6, 0.9 Hz, 1H), 7.32 (td, J=7.8, 1.5 Hz, 1H), 7.26 (d, J=8.1 Hz, 1H), 7.13 (d, J=8.3 Hz, 1H), 6.74 (s, 1H), 5.23 (s, 2H).

¹³C NMR (101 MHz, DMSO) δ 176.70, 161.44, 157.42, 157.36, 151.31, 135.75, 135.24, 132.13, 129.52, 129.13, 127.82, 121.01, 115.17, 114.63, 110.80, 108.71, 69.71

m. p.: 244.3° C.

MS (ESI) m/z 373 (⁷⁹Br), 375 (⁸¹Br) [M−H]⁻.

### EXAMPLE 6

### Preparation of 5-((4-bromobenzyl)oxy)-4-oxo-4H-chromene-2-carboxylic Acid (4b)

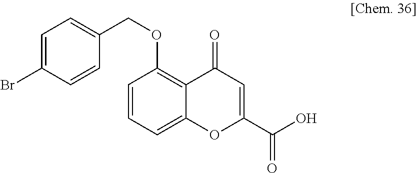 [SMILES: O=C(O)c1cc(=O)c2c(OCc3ccc(Br)cc3)cccc2o1]

The crude was prepared according to general procedure C starting from 3b (1.586 g, 3.93 mmol) and purified by trituration in methanol and washed with diethyl ether to give 4b (1.259, 85%). The desired product can be crystallised in methanol to give white crystals. C₁₇H₁₁BrO₅.

¹H NMR (400 MHz, DMSO) δ 7.76 (t, J=8.4 Hz, 1H), 7.66-7.59 (m, 4H), 7.24 (dd, J=8.4, 0.7 Hz, 1H), 7.14-7.10 (m, 1H), 6.76 (s, 1H), 5.27 (s, 2H)

¹³C NMR (101 MHz, DMSO) δ 176.69, 161.44, 157.69, 157.35, 151.15, 136.27, 135.14, 131.20, 128.89, 120.58, 115.21, 114.69, 110.58, 108.93, 69.23

m. p.: 204.6-205.3° C.

MS (ESI) m/z 374 (⁷⁹Br), 376 (⁸¹Br) [M]⁺.

### EXAMPLE 7

### Preparation of methyl (5-((2-bromobenzyl)oxy)-4-oxo-4H-chromene-2-carbonylamino)-L-alloisoleucinate (5a)

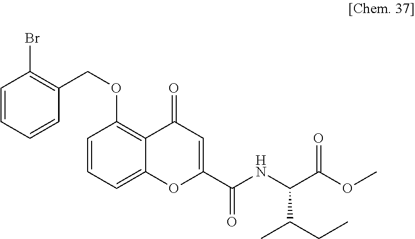 [SMILES: CCC(C)[C@H](NC(=O)c1cc(=O)c2c(OCc3ccccc3Br)cccc2o1)C(=O)OC]

The crude was prepared according to general procedure D starting from 4a (511 mg, 1.36 mmol) and isoleucine methyl ester hydrochloride (0.485 g, 2.72 mmol) and purified by recrystallisation in methanol to give 5a (0.352 g, 51%). C₂₄H₂₄BrNO₆.

¹H NMR (400 MHz, DMSO) δ 9.23 (d, J=7.7 Hz, 1H), 8.14 (d, J=7.4 Hz, 1H), 7.82 (t, J=8.4 Hz, 1H), 7.69 (d, J=7.9 Hz, 1H), 7.52 (t, J=7.4 Hz, 1H), 7.40 (d, J=8.4 Hz, 1H), 7.33 (t, J=7.3 Hz, 1H), 7.17 (d, J=8.3 Hz, 1H), 6.74 (s, 1H), 5.25 (s, 2H), 4.39 (t, J=7.7 Hz, 1H), 3.70 (s, 3H), 2.10-1.99 (m, 1H), 1.60-1.48 (m, J=11.6, 5.8 Hz, 1H), 1.33-1.23 (m, 1H), 0.97-0.86 (m, 6H)

¹³C NMR (101 MHz, DMSO) δ 176.44, 171.41, 159.61, 157.38, 157.05, 152.97, 135.76, 135.11, 132.17, 129.56, 129.14, 127.85, 121.04, 114.49, 112.75, 111.09, 108.81, 69.70, 57.26, 51.90, 35.48, 25.09, 15.37, 10.77

m. p.: 123.1-125.7° C.

HRMS (ESI/QTOF):
calculated for C₂₄H₂₅BrNO₆ (M+H⁺): 502.0865,
found: 502.0860.

Purity (HPLC)>95%.

### EXAMPLE 8

### Preparation of methyl (5-((4-bromobenzyl)oxy)-4-oxo-4H-chromene-2-carbonylamino)-L-alloisoleucinate (5b)

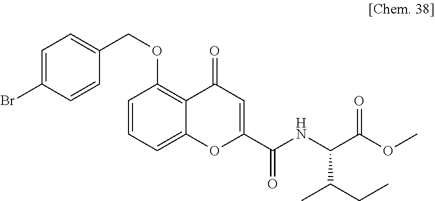 [SMILES: CCC(C)[C@H](NC(=O)c1cc(=O)c2c(OCc3ccc(Br)cc3)cccc2o1)C(=O)OC]

The crude was prepared according to general procedure D starting from 4b (300 mg, 0.80 mmol) and L-isoleucine methyl ester hydrochloride (290 mg, 1.60 mmol) and purified by recrystallisation in methanol to afford 5b (318 mg, 79%). C₂₄H₂₄BrNO₆.

¹H NMR (400 MHz, DMSO) δ 9.17 (d, J=7.9 Hz, 1H), 7.77 (t, J=8.4 Hz, 1H), 7.64-7.56 (m, 4H), 7.34 (dd, J=8.5, 0.7 Hz, 1H), 7.12 (d, J=8.1 Hz, 1H), 6.70 (s, 1H), 5.25 (s, 2H), 4.37 (t, J=7.7 Hz, 1H), 3.68 (s, 3H), 2.07-1.96 (m, 1H), 1.58-1.45 (m, 1H), 1.32-1.19 (m, 1H), 0.95-0.84 (m, 6H)

¹³C NMR (101 MHz, DMSO) δ 176.41, 171.40, 159.62, 157.63, 157.04, 152.91, 136.29, 134.95, 131.21, 128.92, 120.59, 114.54, 112.73, 110.86, 108.99, 69.19, 57.24, 51.88, 35.48, 25.09, 15.37, 10.77

### EXAMPLE 9

### Preparation of methyl (5-((2-bromobenzyl)oxy)-4-oxo-4H-chromene-2-carbonylamino)-L-leucinate (5c)

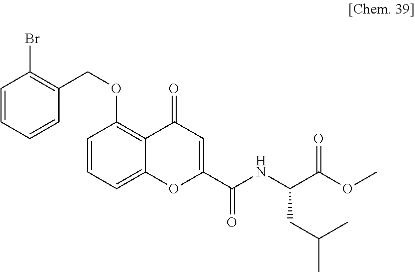 [SMILES: COC(=O)[C@H](CC(C)C)NC(=O)c1cc(=O)c2c(OCc3ccccc3Br)cccc2o1]

The crude was prepared according to general procedure D starting from 4a (500 mg, 1.33 mmol) and L-leucine methyl ester hydrochloride (0.483 g, 2.66 mmol) and purified by recrystallisation in methanol to give 5c (234 mg, 35%). C₂₄H₂₄BrNO₆.

¹H NMR (400 MHz, CDCl₃) δ 8.13 (d, J=7.4 Hz, 1H), 7.57 (t, J=8.3 Hz, 1H), 7.48 (d, J=7.9 Hz, 1H), 7.38 (t, J=7.5 Hz, 1H), 7.18-7.05 (m, 3H), 6.98 (s, 1H), 6.90 (d, J=8.3 Hz, 1H), 5.18 (s, 2H), 4.82-4.73 (m, 1H), 3.74 (s, 3H), 1.80-1.60 (m, 3H), 0.99-0.87 (m, 6H).

¹³C NMR (101 MHz, CDCl₃) δ 177.52, 172.93, 159.05, 158.46, 157.33, 152.34, 135.55, 134.74, 132.10, 129.07, 128.79, 128.12, 120.82, 115.42, 114.21, 110.63, 108.66, 70.38, 52.73, 51.14, 41.81, 25.02, 22.82, 22.05

m. p.: 60.3-60.4° C.

HRMS (ESI/QTOF):
calculated for C₂₄H₂₅BrNO₆ (M+H⁺): 502.0865,
found: 502.0865.

Purity (HPLC)>98%.

### EXAMPLE 10

### Preparation of methyl (5-((2-bromobenzyl)oxy)-4-oxo-4H-chromene-2-carbonylamino)-L-valinate (5d)

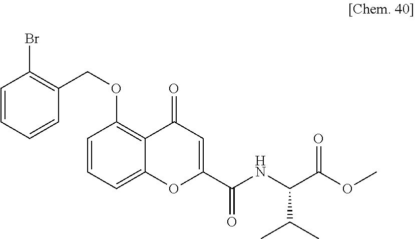 [SMILES: COC(=O)[C@@H](NC(=O)c1cc(=O)c2c(OCc3ccccc3Br)cccc2o1)C(C)C]

The crude was prepared according to general procedure D starting from 4a (0.200 g, 0.53 mmol) and L-valine methyl ester hydrochloride (0.179 g, 1.07 mmol) and purified by recrystallisation in methanol to afford 5d (0.126 g, 48%). C₂₃H₂₂BrNO₆.

¹H NMR (400 MHz, DMSO) δ 9.20 (d, J=7.8 Hz, 1H), 8.12 (d, J=7.4 Hz, 1H), 7.82 (t, J=8.4 Hz, 1H), 7.69 (d, J=7.8 Hz, 1H), 7.51 (t, J=7.5 Hz, 1H), 7.39 (d, J=8.4 Hz, 1H), 7.33 (t, J=7.4 Hz, 1H), 7.16 (d, J=8.3 Hz, 1H), 6.74 (s, 1H), 5.24 (s, 2H), 4.33 (t, J=7.7 Hz, 1H), 3.70 (s, 3H), 2.32-2.19 (m, 1H), 1.06-0.92 (m, 6H)

¹³C NMR (101 MHz, DMSO) δ 176.42, 171.34, 159.69, 157.41, 157.07, 153.03, 135.76, 135.09, 132.17, 129.57, 129.18, 127.84, 121.07, 114.53, 112.74, 111.10, 108.88, 69.76, 58.56, 51.90, 29.46, 19.04, 18.95

m. p.: 127.0-128.2° C.

HRMS (ESI/QTOF):
calculated for C₂₃H₂₃BrNO₆ (M+H⁺): 488.0709,
found: 488.0719.

Purity (HPLC)>98%.

### EXAMPLE 11

### Preparation of methyl (5-((2-bromobenzyl)oxy)-4-oxo-4H-chromene-2-carbonylamino)-L-phenylalaninate (5e)

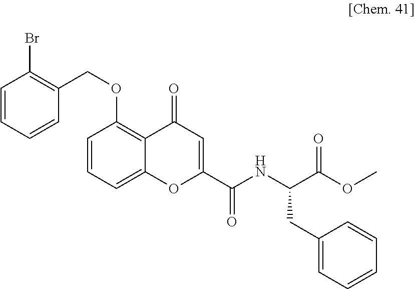 [SMILES: COC(=O)[C@H](Cc1ccccc1)NC(=O)c1cc(=O)c2c(OCc3ccccc3Br)cccc2o1]

The crude was prepared according to general procedure D starting from 4a (0.194 g, 0.52 mmol) and L-phenylalanine methyl ester hydrochloride (0.223 g, 1.04 mmol) and purified by precipitation in diethyl ether and cyclohexane to give 5e (0.187 g, 70%). C₂₇H₂₂BrNO₆.

¹H NMR (400 MHz, DMSO) δ 9.47 (s, 1H), 8.13 (dd, J=7.7, 1.5 Hz, 1H), 7.82 (t, J=8.4 Hz, 1H), 7.68 (dd, J=8.0, 1.1 Hz, 1H), 7.51 (td, J=7.6, 1.1 Hz, 1H), 7.35-7.27 (m, 6H), 7.25-7.19 (m, 1H), 7.16 (d, J=8.0 Hz, 1H), 6.64 (s, 1H), 5.24 (s, 2H), 4.77-4.71 (m, 1H), 3.69 (s, 3H), 3.26 (dd, J=13.8, 5.4 Hz, 1H), 3.21-3.13 (m, 1H).

¹³C NMR (101 MHz, DMSO) δ 176.33, 171.26, 159.21, 157.42, 156.95, 152.77, 137.22, 135.74, 135.24, 132.15, 129.54, 129.11, 129.06, 128.31, 127.84, 126.62, 121.01, 114.43, 112.57, 110.82, 108.85, 69.69, 54.13, 52.20, 35.93, 30.67

m. p.: 145.5-147.5° C.

HRMS (ESI/QTOF)
calculated for C₂₇H₂₃BrNO₆ (M+H⁺): 536.0709,
found: 536.0711.

Purity (HPLC)>97%.

### EXAMPLE 12

### Preparation of methyl (5-((4-bromobenzyl)oxy)-4-oxo-4H-chromene-2-carbonylamino)-L-phenylalaninate (5f)

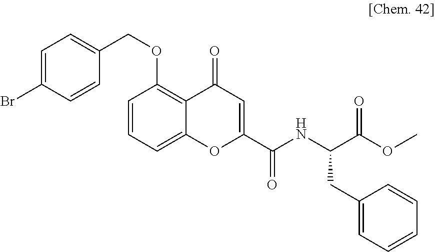 [SMILES: COC(=O)[C@H](Cc1ccccc1)NC(=O)c1cc(=O)c2c(OCc3ccc(Br)cc3)cccc2o1]

The crude was prepared according to general procedure D starting from 4b (0.472 g, 1.26 mmol) and L-phenylalanine methyl ester hydrochloride (0.453 g, 2.52 mmol) and purified by recrystallisation in methanol to give 5f (0.551 g, 90%). C₂₇H₂₂BrNO₆.

¹H NMR (400 MHz, DMSO) δ 9.43 (d, J=7.9 Hz, 1H), 7.79 (t, J=8.4 Hz, 1H), 7.68-7.54 (m, 4H), 7.30 (d, J=12.6 Hz, 5H), 7.23 (d, J=6.1 Hz, 1H), 7.13 (d, J=8.3 Hz, 1H), 6.63 (s, 1H), 5.26 (s, 2H), 4.73 (dd, J=13.5, 9.1 Hz, 1H), 3.68 (s, 3H), 3.26 (dd, J=13.8, 5.2 Hz, 1H), 3.16 (dd, J=13.6, 10.3 Hz, 1H).

¹³C NMR (101 MHz, DMSO) δ 176.31, 171.22, 159.22, 157.68, 156.94, 152.71, 137.20, 136.26, 135.08, 131.20, 129.05, 128.91, 128.31, 126.62, 120.59, 114.50, 112.56, 110.60, 109.06, 69.21, 54.10, 52.20, 35.93

Decomposition point: 153.1° C.

HRMS (ESI/QTOF)
calculated for C₂₇H₂₃BrNO₆ (M+H⁺): 536.0709,
found: 536.0704.

Purity (HPLC)>96%.

### EXAMPLE 13

### Preparation of methyl (5-((2-bromobenzyl)oxy)-4-oxo-4H-chromene-2-carbonylamino)-L-tryptophanate (5 g)

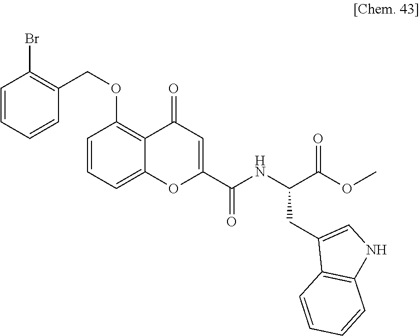 [SMILES: COC(=O)[C@H](Cc1c[nH]c2ccccc12)NC(=O)c1cc(=O)c2c(OCc3ccccc3Br)cccc2o1]

The crude was prepared according to general procedure D starting from 4a (0.100 g, 0.27 mmol) and L-tryptophan methyl ester hydrochloride (0.136 g, 0.53 mmol) and purified by recrystallisation in methanol to give 5 g (0.043 g, 28%). C₂₉H₂₃BrN₂O₆.

¹H NMR (400 MHz, DMSO) δ 10.89 (s, 2H), 9.36 (d, J=7.8 Hz, 2H), 8.12 (d, J=7.7 Hz, 2H), 7.83 (t, J=8.4 Hz, 2H), 7.69 (dd, J=8.0, 0.9 Hz, 2H), 7.62 (d, J=7.8 Hz, 2H), 7.51 (td, J=7.5, 0.9 Hz, 2H), 7.39-7.28 (m, 6H), 7.25 (d, J=2.2 Hz, 2H), 7.17 (d, J=8.3 Hz, 2H), 7.07 (td, J=7.7, 0.9 Hz, 2H), 6.98 (t, J=7.4 Hz, 2H), 6.65 (s, 2H), 5.24 (s, 4H), 4.80-4.70 (m, 2H), 3.69 (s, 7H)

¹³C NMR (101 MHz, DMSO) δ 176.36, 171.52, 159.18, 157.43, 156.95, 152.81, 136.09, 135.74, 135.21, 132.17, 129.57, 129.15, 127.85, 127.03, 123.79, 121.05, 121.02, 118.44, 118.04, 114.43, 112.55, 111.48, 110.83, 109.47, 108.89, 69.73, 53.80, 52.19, 26.38 Two proton signals below the water peak.

m. p.: 250-252.8° C.

HRMS (ESI/QTOF):
calculated for C₂₉H₂₄BrN₂O₆ (M+H⁺): 575.0818,
found: 575.0821.

Purity (HPLC)>99%.

### EXAMPLE 14

### Preparation of methyl (5-((4-bromobenzyl)oxy)-4-oxo-4H-chromene-2-carbonylamino)-L-tryptophanate (5h)

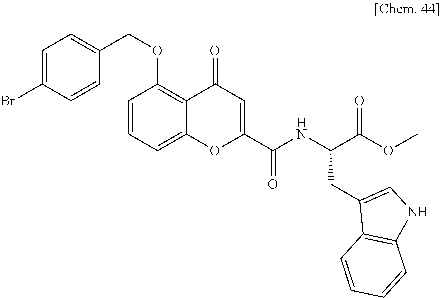 [SMILES: COC(=O)[C@H](Cc1c[nH]c2ccccc12)NC(=O)c1cc(=O)c2c(OCc3ccc(Br)cc3)cccc2o1]

The crude was prepared according to general procedure D starting from 4b (0.100 g, 0.27 mmol) and L-tryptophan methyl ester hydrochloride (0.136 g, 0.533 mmol) and purified by recrystallisation in methanol to afford 5h (0.126 g, 82%). C₂₉H₂₃BrN₂O₆.

¹H NMR (400 MHz, DMSO) δ 10.90 (s, 1H), 9.34 (d, J=7.8 Hz, 1H), 7.79 (t, J=8.4 Hz, 1H), 7.69-7.52 (m, 5H), 7.35 (d, J=8.1 Hz, 1H), 7.29 (d, J=8.1 Hz, 1H) 7.25 (d, J=2.2 Hz, 1H), 7.12 (d, J=8.3 Hz, 1H), 7.10-7.03 (m, 1H), 7.02-6.94 (m, 1H), 6.63 (s, 1H), 5.25 (s, 2H), 4.80-4.69 (m, 1H), 3.68 (s, 3H)

¹³C NMR (101 MHz, DMSO) δ 176.34, 171.50, 159.19, 157.67, 156.92, 152.76, 136.25, 136.10, 135.04, 131.20, 128.92, 127.03, 123.78, 121.02, 120.59, 118.44, 118.04, 114.48, 112.54, 111.48, 110.61, 109.47, 109.06, 69.23, 53.79, 52.18, 26.39 Two proton signals below the water peak.

m. p.: 235.3-236.2° C.

HRMS (ESI/QTOF):

calculated for C₂₉H₂₄BrN₂O₆ (M+H⁺): 575.0818.

found: 575.0807.

Purity (HPLC)>99%.

### EXAMPLE 15

### Preparation of methyl (5-((4-bromobenzyl)oxy)-4-oxo-4H-chromene-2-carbonylamino)-D-tryptophanate (5i)

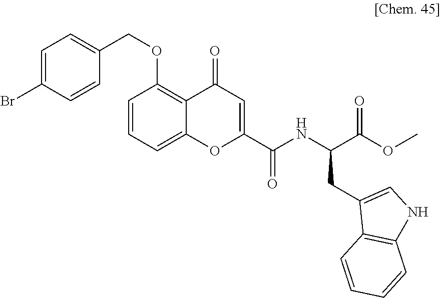 [SMILES: COC(=O)[C@@H](Cc1c[nH]c2ccccc12)NC(=O)c1cc(=O)c2c(OCc3ccc(Br)cc3)cccc2o1]

The crude was prepared according to general procedure D starting from 4b (0.300 g, 0.80 mmol) and D-tryptophan methyl ester hydrochloride (0.408 g, 1.60 mmol) and purified by precipitation in ethyl acetate and cyclohexane to afford 5i (0.375 g, 81%). C₂₉H₂₃BrN₂O₆.

¹H NMR (400 MHz, DMSO) δ 10.90 (s, 1H), 9.34 (d, J=7.8 Hz, 1H), 7.79 (t, J=8.4 Hz, 1H), 7.66-7.57 (m, 5H), 7.35 (d, J=8.1 Hz, 1H), 7.29 (d, J=8.3 Hz, 1H), 7.25 (d, J=2.1 Hz, 1H) 7.12 (d, J=8.3 Hz, 1H), 7.07 (t, J=7.2 Hz, 1H), 6.98 (t, J=7.4 Hz, 1H), 6.63 (s, 1H), 5.25 (s, 2H), 4.79-4.70 (m, 1H), 3.69 (s, 3H), 3.43-3.38 (m, 1H), 3.33-3.27 (m, 1H)

¹³C NMR (101 MHz, DMSO) δ 176.33, 171.51, 159.18, 157.67, 156.93, 152.75, 136.26, 136.10, 135.04, 131.20, 128.91, 127.03, 123.78, 121.01, 120.59, 118.43, 118.04, 114.48, 112.54, 111.48, 110.60, 109.47, 109.04, 69.21, 53.79, 52.18, 26.38

m. p.: 236.2-237.9° C.

HRMS (ESI/QTOF):
calculated for C₂₉H₂₄BrN₂O₆ (M+H⁺): 575.0818,
found: 575.0822.

Purity (HPLC)>99%.

### EXAMPLE 16

### Preparation of (5-((2-bromobenzyl)oxy)-4-oxo-4H-chromene-2-carbonylamino)-L-alloisoleucine (6a)

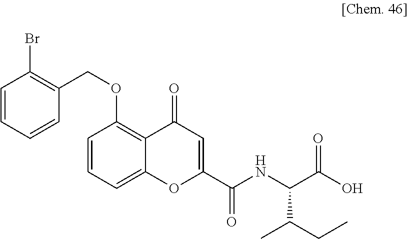 [SMILES: CCC(C)[C@H](NC(=O)c1cc(=O)c2c(OCc3ccccc3Br)cccc2o1)C(=O)O]

The crude was prepared according to general procedure E starting from 5a (0.256 g, 0.51 mmol) and purified by recrystallisation in methanol to give 6a (0.114 g, 46%). C₂₃H₂₂BrNO₆.

¹H NMR (400 MHz, DMSO) δ 8.99 (d, J=8.1 Hz, 1H), 8.14 (d, J=7.5 Hz, 1H), 7.82 (t, J=8.4 Hz, 1H), 7.69 (dd, J=7.9, 0.8 Hz, 1H), 7.52 (td, J=7.6, 0.8 Hz, 1H), 7.41 (d, J=8.2 Hz, 1H), 7.33 (td, J=7.8, 1.5 Hz, 1H), 7.17 (d, J=8.2 Hz, 1H), 6.73 (s, 1H), 5.25 (s, 2H), 4.39-4.31 (m, 1H), 2.08-1.98 (m, 1H), 1.61-1.48 (m, 1H), 1.35-1.23 (m, 1H), 0.97 (d, J=6.8 Hz, 3H), 0.90 (t, J=7.4 Hz, 3H).

¹³C NMR (101 MHz, DMSO) δ 176.46, 172.23, 159.43, 157.39, 157.06, 153.18, 135.76, 135.07, 132.16, 129.57, 129.16, 127.84, 121.06, 114.49, 112.64, 111.12, 108.84, 69.73, 57.21, 35.62, 25.04, 15.52, 10.96

m. p.: 242.8-243.7° C.

HRMS (ESI/QTOF):
calculated for C₂₃H₂₃BrNO₆ (M+H⁺): 488.0709,
found: 488.0712.

Purity (HPLC)>98%.

### EXAMPLE 17

### Preparation of (5-((4-bromobenzyl)oxy)-4-oxo-4H-chromene-2-carbonyl)-L-alloisoleucine (6b)

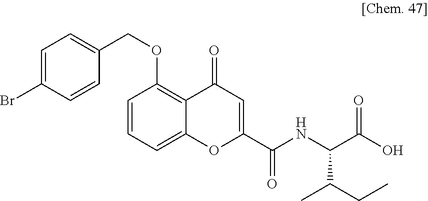 [SMILES: CCC(C)[C@H](NC(=O)c1cc(=O)c2c(OCc3ccc(Br)cc3)cccc2o1)C(=O)O]

The crude was prepared according to general procedure E starting from 5b (671 mg, 0.51 mmol) and purified by recrystallisation in methanol to give 6b (419 mg, 64%). C₂₃H₂₂BrNO₆.

¹H NMR (400 MHz, DMSO) δ 12.91 (s, 1H), 8.98 (d, J=8.1 Hz, 1H), 7.78 (t, J=8.4 Hz, 1H), 7.67-7.57 (m, 4H), 7.37 (dd, J=8.5, 0.6 Hz, 1H), 7.13 (d, J=8.2 Hz, 1H) 6.72 (s, 1H), 5.26 (s, 2H), 4.40-4.29 (m, 1H), 2.07-1.96 (m, 1H), 1.61-1.45 (m, 1H), 1.38-1.20 (m, 1H), 0.96 (d, J=6.8 Hz, 3H), 0.90 (t, J=7.4 Hz, 3H). (ERO1-94)

¹³C NMR (101 MHz, DMSO) δ 176.44, 172.23, 159.44, 157.63, 157.04, 153.12, 136.28, 134.91, 131.20, 128.93, 120.59, 114.54, 112.63, 110.89, 108.99, 69.21, 57.19, 35.63, 25.04, 15.52, 10.96

p.f.: 230.1-230.4° C.

HRMS (ESI/QTOF)
calculated for C₂₃H₂₃BrNO₆ (M+H⁺): 488.0709,
found: 488.0715.

Purity (HPLC)>98%.

### EXAMPLE 18

### Preparation of (5-((2-bromobenzyl)oxy)-4-oxo-4H-chromene-2-carbonylamino)-L-phenylalanine (6c)

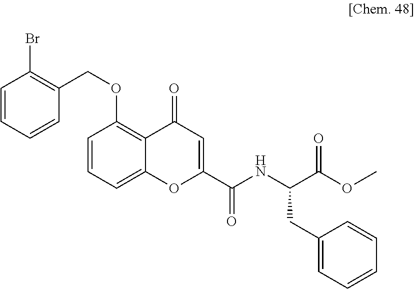 [SMILES: COC(=O)[C@H](Cc1ccccc1)NC(=O)c1cc(=O)c2c(OCc3ccccc3Br)cccc2o1]

The crude was prepared according to general procedure E starting from 5e (0.320 g, 0.60 mmol) and purified by recrystallisation in methanol to give 6c (0.210 g, 67%). C₂₆H₂₀BrNO₆.

¹H NMR (400 MHz, DMSO) δ 9.29 (d, J=8.2 Hz, 2H), 8.11 (dd, J=7.6, 1.0 Hz, 2H), 7.82 (t, J=8.4 Hz, 2H), 7.68 (dd, J=7.9, 0.8 Hz, 2H), 7.50 (td, J=7.6, 0.9 Hz, 2H), 7.37-7.25 (m, 11H), 7.24-7.10 (m, 4H), 6.63 (s, 2H), 5.23 (s, 4H), 4.72-4.58 (m, 2H), 3.14 (dd, J=13.8, 10.3 Hz, 5H) A peak below the water signal.

¹³C NMR (101 MHz, DMSO) δ 176.37, 172.20, 159.04, 157.44, 156.96, 153.00, 137.72, 135.73, 135.20, 132.16, 129.57, 129.17, 129.02, 128.25, 127.83, 126.47, 121.06, 114.45, 112.43, 110.85, 108.91, 69.75, 54.18, 36.01

p.f.: 240.4-241.7° C.

HRMS (ESI/QTOF)
calculated for C₂₆H₂₁BrNO₆ (M+H⁺): 522.0552,
found: 522.0558.

### EXAMPLE 19

### Preparation of (5-((4-bromobenzyl)oxy)-4-oxo-4H-chromene-2-carbonylamino)-L-phenylalanine (6d)

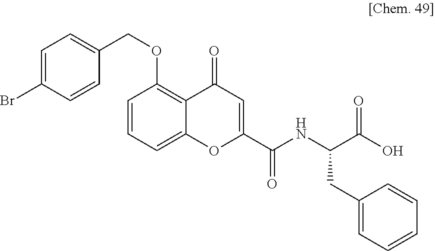 [SMILES: O=C(N[C@@H](Cc1ccccc1)C(=O)O)c1cc(=O)c2c(OCc3ccc(Br)cc3)cccc2o1]

The crude was prepared according to general procedure E starting from 5f (0.551 g, 1.03 mmol) and purified by recrystallisation in methanol to give 6d (0.446 g, 83%). C₂₆H₂₀BrNO₆.

¹H NMR (400 MHz, DMSO) δ 8.51 (d, J=6.8 Hz, 1H), 7.72 (t, J=8.4 Hz, 1H), 7.66-7.57 (m, 4H), 7.24-7.16 (m, 5H), 7.16-7.07 (m, 2H), 6.62 (s, 1H), 5.24 (s, 2H), 4.24 (dd, J=11.0, 5.9 Hz, 1H), 3.26 (dd, J=13.5, 4.6 Hz, 1H), 3.11 (dd, J=13.5, 6.8 Hz, 1H) (ERO1-71)

¹³C NMR (101 MHz, DMSO) δ 176.35, 172.19, 159.11, 157.67, 156.94, 152.89, 137.66, 136.26, 135.06, 131.20, 129.02, 128.91, 128.27, 126.50, 120.58, 114.47, 112.44, 110.62, 109.04, 69.21, 54.08, 35.94

Decomposition point: 231.5° C.

HRMS (ESI/QTOF):
calculated for C₂₆H₂₁BrNO₆ ((M+H⁺): 522.0552,
found: 522.0559.

Purity (HPLC)>95%.

### EXAMPLE 20

### Preparation of (5-((4-bromobenzyl)oxy)-4-oxo-4H-chromene-2-carbonylamino)-D-tryptophan (6e)

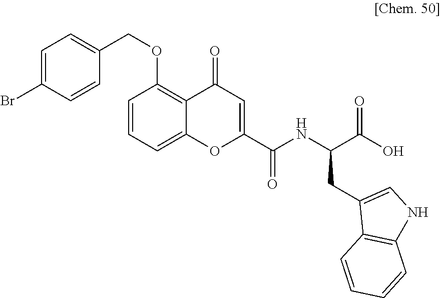 [SMILES: O=C(N[C@H](Cc1c[nH]c2ccccc12)C(=O)O)c1cc(=O)c2c(OCc3ccc(Br)cc3)cccc2o1]

The crude was prepared according to general procedure E starting from 5i (0.250 g, 0.27 mmol) and purified by precipitation in ethyl acetate and cyclohexane to afford 6e (0.158 g, 66%). C₂₈H₂₁BrN₂O₆.

¹H NMR (400 MHz, DMSO) δ 10.87 (d, J=1.4 Hz, 1H), 9.18 (d, J=8.1 Hz, 1H), 7.79 (t, J=8.4 Hz, 1H), 7.69-7.56 (m, 5H), 7.34 (d, J=8.1 Hz, 1H) J=8.2 Hz, 1H), 7.24 (d, J=2.2 Hz, 1H), 7.13 (d, J=8.3 Hz, 1H), 7.10-7.04 (m, 1H), 7.01-6.95 (m, 1H), 6.63 (s, 1H), 5.25 (s, 2H), 4.73-4.65 (m, 1H) Two signals below the water peak.

¹³C NMR (101 MHz, DMSO) δ 176.37, 172.52, 159.06, 157.66, 156.92, 152.92, 136.26, 136.08, 135.04, 131.20, 128.91, 127.09, 123.66, 120.98, 120.58, 118.39, 118.16, 114.44, 112.42, 111.43, 110.59, 109.93, 109.00, 69.18, 53.73, 26.37 Two proton signals below the water peak.

m. p.: 185.5-186.7° C.

HRMS (ESI/QTOF):
calculated for C₂₈H₂₂BrN₂O₆ (M+H⁺): 561.0661,
found: 561.0672.

Purity (HPLC)>99%.

### EXAMPLES 21 TO 27

### Series 2

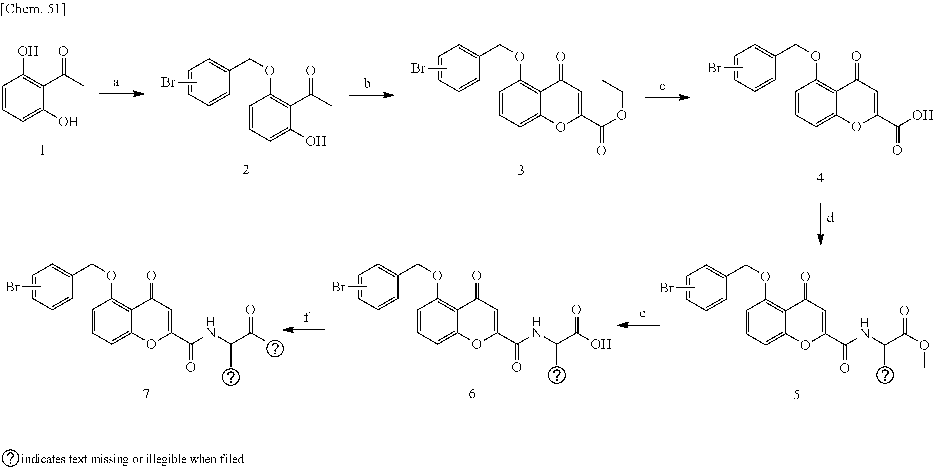

Note: In the following protocols, “molecule number+a” refers to when the bromine is in position 2 of the aromatic ring while “molecule number+b” refers to when the bromine is in position 4 of the aromatic ring.

Note: same synthesis scheme as series 1 up to step e. Only step f allowing access to compounds 7 is different.

General Operating Procedure F

To a solution of carboxylic acid derivative 6 (1 equiv) in anhydrous DMF (20 mL/mmol) was added TBTU (1.5 equiv or 2 equiv). The solution was stirred for 30 minutes at room temperature. Thus, a solution of amino acid derivative (1.5 or 2 equiv) in DMF (10 mL/mmol) in the presence of DIEA (5 equiv) was added carefully to the previous one. The reaction was stirred for 24 h at room temperature and monitored by TLC (cyclohexane/ethyl acetate 3:2). The solution was poured into acidified water (1M HCl) and extracted with ethyl acetate. The organic phases were collected and washed with a 20% NaHCO₃ solution and brine before being dried over magnesium sulphate, filtered and evaporated in vacuo.

### EXAMPLE 21

### Preparation of methyl (5-((2-bromobenzyl)oxy)-4-oxo-4H-chromene-2-carbonylamino)-L-alloisoleucyl-L-valinate (7a)

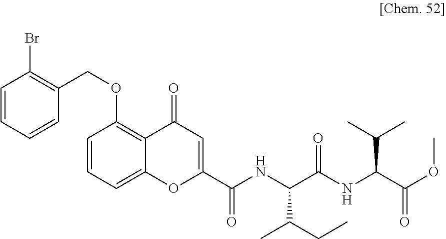 [SMILES: CCC(C)[C@H](NC(=O)c1cc(=O)c2c(OCc3ccccc3Br)cccc2o1)C(=O)N[C@H](C(=O)OC)C(C)C]

The crude was prepared according to general procedure F starting from 6a (0.091 g, 0.19 mmol), L-valine methyl ester (0.064 g, 0.38 mmol) and purified by precipitation in ethyl acetate and a minimal amount of cyclohexane, followed by washing with diethyl ether to provide 7a (0.042 g, 37%). C₂₉H₃₃BrN₂O₇.

¹H NMR (400 MHz, DMSO) δ 8.79 (d, J=8.4 Hz, 0.5H), 8.60-8.50 (m, 1H), 8.45 (d, J=7.2 Hz, 0.5H), 8.13 (d, J=7.5 Hz, 1H), 7.81 (t, J=8.0 Hz, 1H), 7.69 (d, J=7.8 Hz, 1H), 7.52 (t, J=7.3 Hz, 1H), 7.40 (d, J=8.1 Hz, 1H), 7.33 (t, J=7.3 Hz, 1H), 7.16 (d, J=8.4 Hz, 1H), 6.72 (d, J=5.0 Hz, 1H), 5.25 (s, 2H), 4.73-4.65 (m, 0.5H), 4.54-4.45 (m, 0.5H), 4.27-4.21 (m, 0.5H), 4.21-4.14 (m, 0.5H), 3.65 (d, J=4.7 Hz, 3H), 2.13-1.92 (m, 2H), 1.64-1.47 (m, 1H), 1.28-1.14 (m, 1H), 1.04-0.70 (m, 12H).

¹³C NMR (101 MHz, DMSO) δ 172.31, 171.91, 171.88, 157.84, 157.83, 157.82, 157.51, 157.49, 157.46, 157.43, 157.42, 153.69, 153.67, 153.65, 135.92, 132.79, 130.34, 129.78, 128.33, 121.94, 114.74, 114.72, 114.70, 114.66, 112.89, 112.87, 112.69, 111.52, 111.46, 109.48, 109.46, 109.37, 109.35, 70.33, 70.31, 58.31, 58.30, 58.21, 58.05, 58.02, 57.96, 52.33, 52.26, 52.23, 52.21, 36.32, 30.00, 25.02, 19.31, 19.17, 19.13, 18.66, 18.59, 15.29, 15.28, 10.85.

HRMS (ESI/QTOF)
calculated for C₂₉H₃₄BrN₂O₇ (M+H⁺): 601.1549,
found: 601.1533.

### EXAMPLE 22

### Preparation of methyl (5-((2-bromobenzyl)oxy)-4-oxo-4H-chromene-2-carbonylamino)-L-alloisoleucyl-L-leucinate (7b)

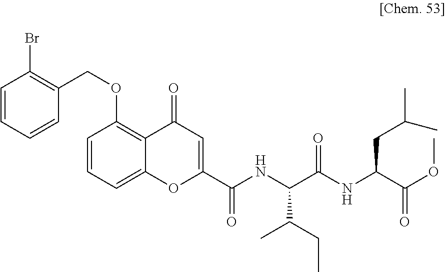 [SMILES: CCC(C)[C@H](NC(=O)c1cc(=O)c2c(OCc3ccccc3Br)cccc2o1)C(=O)N[C@@H](CC(C)C)C(=O)OC]

The crude was prepared according to general procedure F starting from 6a (0.082 mg, 0.16 mmol), L-leucine methyl ester (0.061 g, 0.32 mmol) and purified by precipitation in ethyl acetate and a minimal amount of cyclohexane, followed by washing with diethyl ether to afford 7b (0.030 g, 31%). C₃₀H₃₅BrN₂O₇.

¹H NMR (400 MHz, DMSO) δ 8.79 (d, J=8.8 Hz, 0.5H), 8.64 (d, J=7.6 Hz, 1H), 8.56 (d, J=7.7 Hz, 0.5H), 8.13 (d, J=7.6 Hz, 1H), 7.81 (t, J=8.2 Hz, 1H), 7.69 (d, J=8.0 Hz, 1H), 7.52 (t, J=7.5 Hz, 1H), 7.40 (dd, J=8.3, 4.4 Hz, 1H), 7.33 (t, J=7.5 Hz, 1H), 7.16 (d, J=8.1 Hz, 1H), 6.73 (d, J=7.3 Hz, 1H), 5.25 (s, 2H), 4.64-4.56 (m, 0.5H), 4.41 (t, J=8.7 Hz, 1H), 4.37-4.27 (m, 0.5H), 3.63 (s, 1H), 2.05-1.92 (m, 1H), 1.74-1.43 (m, 4H), 1.27-1.13 (m, 1H), 0.98-0.81 (m, 12H)).

¹³C NMR (126 MHz, DMSO) δ 177.64, 174.72, 173.46, 173.29, 171.55, 171.48, 159.81, 159.64, 157.82, 157.41, 153.65, 153.63, 135.89, 135.84, 132.77, 130.31, 129.75, 128.32, 121.89, 114.67, 112.93, 112.88, 111.50, 109.43, 70.31, 58.05, 57.49, 52.53, 52.42, 52.32, 51.52, 51.02, 50.90, 41.57, 40.89, 37.32, 36.39, 25.92, 24.96, 24.70, 24.64, 23.10, 23.01, 22.99, 21.82, 21.57, 21.44, 21.23, 15.31, 14.95, 11.64, 10.85.

HRMS (ESI/QTOF):
calculated for C₃₀H₃₆BrN₂O₇ (M+H⁺): 615.1690,
found: 615.1684.

### EXAMPLE 23

### Preparation of methyl (5-((4-bromobenzyl)oxy)-4-oxo-4H-chromene-2-carbonylamino)-L-alloisoleucyl-L-valinate (7c)

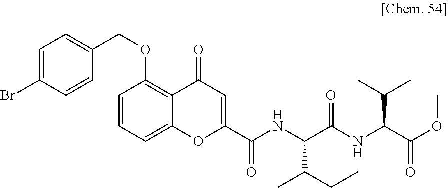 [SMILES: CCC(C)[C@H](NC(=O)c1cc(=O)c2c(OCc3ccc(Br)cc3)cccc2o1)C(=O)N[C@H](C(=O)OC)C(C)C]

The crude was prepared according to general procedure F starting from 6a (0.123 g, 0.25 mmol), L-valine methyl ester (0.084 g, 0.50 mmol) and purified by trituration in diethyl ether to afford 7c (0.061 g, 41%). C₂₉H₃₃BrN₂O₇.

¹H NMR (400 MHz, DMSO) δ 8.76 (d, J=8.6 Hz, 0.5H), 8.54 (d, J=9.4 Hz, 1H), 8.43 (d, J=7.5 Hz, 0.5H), 7.76 (t, J=8.3 Hz, 1H), 7.60 (q, J=8.6 Hz, 4H), 7.35 (d, J=8.2 Hz, 1H), 7.11 (d, J=8.3 Hz, 1H), 6.69 (d, J=5.4 Hz, 1H) 2H), 4.67 (dd, J=8.8, 6.9 Hz, 0.5H), 4.48 (t, J=8.8 Hz, 0.5H), 4.22 (dd, J=7.9, 6.9 Hz, 0.5H), 4.15 (t, J=7.0 Hz, 0.5H), 3.63 (d, J=4.9 Hz, 3H), 2.12-1.90 (m, 2H), 1.59-1.36 (m, 1H), 1.25-1.11 (m, 1H), 0.99-0.81 (m, 12H).

¹³C NMR (101 MHz, DMSO) δ 176.43, 171.93, 171.64, 170.88, 170.67, 159.10, 158.94, 157.64, 157.01, 153.21, 136.29, 134.90, 131.19, 128.91, 120.58, 114.52, 112.61, 112.54, 110.83, 109.00, 69.21, 57.66, 57.42, 57.24, 56.72, 51.72, 51.53, 37.09, 35.99, 29.85, 29.58, 25.65, 24.62, 19.02, 18.84, 18.35, 18.20, 14.95, 14.58, 11.37, 10.55.

HRMS (ESI/QTOF):
calculated for C₂₉H₃₄BrN₂O₇ (M+H⁺): 601.1549,
found: 601.1545.

### EXAMPLE 24

### Preparation of methyl (5-((4-bromobenzyl)oxy)-4-oxo-4H-chromene-2-carbonylamino)-L-alloisoleucyl-L-leucinate (7d)

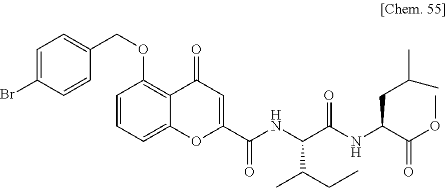 [SMILES: CCC(C)[C@H](NC(=O)c1cc(=O)c2c(OCc3ccc(Br)cc3)cccc2o1)C(=O)N[C@@H](CC(C)C)C(=O)OC]

The crude was prepared according to general procedure F starting from 6b (0.123 g, 0.25 mmol), L-leucine methyl ester (0.091 mg, 0.50 mmol) and purified by trituration in diethyl ether to afford 7d (0.052 g, 34%). C₃₀H₃₅BrN₂O₇.

¹H NMR (400 MHz, DMSO) δ 8.76 (d, J=8.7 Hz, 0.5H), 8.62 (d, J=7.8 Hz, 0.5H), 8.54 (dd, J=8.0, 4.5 Hz, 1H), 7.76 (t, J=8.2 Hz, 1H), 7.66-7.56 (m, 4H), 7.35 (dd, J=8.4, 4.2 Hz, 1H), 7.11 (d, J=8.2 Hz, 1H), 6.70 (d, J=7.5 Hz, 1H), 5.25 (s, 2H), 4.58 (dd, J=8.6, 6.5 Hz, 0.5H), 4.39 (t, J=8.6 Hz, 0.5H), 4.36-4.24 (m, 1H), 3.62 (d, J=2.3 Hz, 3H), 2.03-1.89 (m, 1H), 1.70-1.55 (m, 2H), 1.55-1.45 (m, 2H), 1.24-1.10 (m, 1H), 0.96-0.79 (m, 12H).

¹³C NMR 176.43, 172.81, 172.60, 170.56, 170.42, 159.14, 158.95, 157.63, 157.01, 153.19, 136.28, 134.90, 131.19, 128.91, 120.57, 114.51, 112.62, 112.54, 110.84, 109.00, 69.20, 57.29, 56.64, 51.86, 51.71, 50.39, 50.23, 36.98, 36.00, 25.59, 24.56, 24.23, 24.18, 22.78, 22.66, 21.28, 20.90, 14.99, 14.62, 10.54.

HRMS (ESI/QTOF):
calculated for C₃₀H₃₆BrN₂O₇ (M+H⁺): 615.1690,
found: 615.1690.

### EXAMPLE 25

### Preparation of (S)-5-((2-bromobenzyl)oxy)-N-(1-((2-(5-hydroxy-1H-indol-3-yl)ethyl)amino)-1-oxo-3-phenylpropan-2-yl)-4-oxo-4H-chromene-2-carboxamide (7th)

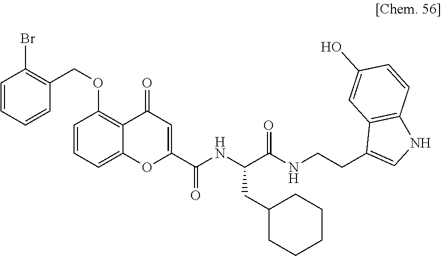 [SMILES: O=C(N[C@@H](CC1CCCCC1)C(=O)NCCc1c[nH]c2ccc(O)cc12)c1cc(=O)c2c(OCc3ccccc3Br)cccc2o1]

The crude was prepared according to general procedure F starting from 6c (0.127 mg, 0.24 mmol), 5-hydroxytryptamine hydrochloride (0.103 mg, 0.48 mmol) and purified by trituration in methanol, followed by washing with diethyl ether to afford 7e (0.063 g, 38%). C₃₆H₃₀BrN₃O₆.

¹H NMR (400 MHz, DMSO) δ 10.49 (d, J=1.6 Hz, 1H), 9.11 (d, J=8.4 Hz, 1H), 8.61 (s, 1H), 8.31 (t, J=5.5 Hz, 1H), 8.10 (dd, J=7.7, 0.8 Hz, 1H), 7.79 (t, J=8.4 Hz, 1H), 7.65 (dd, J=8.0, 0.7 Hz, 1H), 7.48 (td, J=7.6, 0.7 Hz, 1H), 7.37-7.23 (m, 6H), 7.18 (t, J=7.2 Hz, 1H), 7.12 (d, J=8.6 Hz, 2H), 7.04 (d, J=2.1 Hz, 1H), 6.86 (d, J=2.1 Hz, 1H), 6.63 (s, 1H), 6.60 (dd, J=8.6, 2.2 Hz, 1H), 5.21 (s, 2H), 4.73-4.65 (m, 1H), 3.34-3.26 (m, 1H), 3.17 (dd, J=13.7, 4.5 Hz, 1H), 3.05 (dd, J=13.6, 10.2 Hz, 1H), 2.73 (t, J=7.5 Hz, 2H).

¹³C NMR (101 MHz, DMSO) δ 176.46, 170.03, 158.99, 157.41, 156.99, 153.19, 150.16, 137.88, 135.74, 135.14, 132.15, 130.81, 129.55, 129.15, 128.14, 127.83, 126.38, 123.13, 121.06, 114.44, 112.36, 111.65, 111.27, 110.93, 110.64, 108.83, 102.20, 69.74, 54.91, 37.11, 25.14. 31C+2EQ 3C MISSING.

HRMS (ESI/QTOF):
calculated for C₃₆H₃₁BrN₃O₆ (M+H⁺): 680.1396,
found: 680.1392.

### EXAMPLE 26

### Preparation of (S)—N-(1-((2-(1H-indol-3-yl)ethyl)amino)-1-oxo-3-phenylpropan-2-yl)-5-((4-bromobenzyl)oxy)-4-oxo-4H-chromene-2-carboxamide (7f)

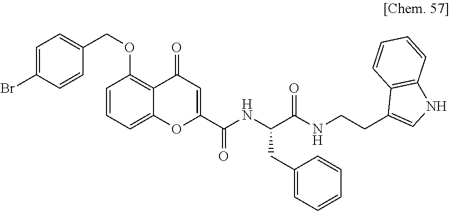 [SMILES: O=C(N[C@@H](Cc1ccccc1)C(=O)NCCc1c[nH]c2ccccc12)c1cc(=O)c2c(OCc3ccc(Br)cc3)cccc2o1]

The crude was prepared according to general procedure F starting from 6d (0.090 g, 0.17 mmol), tryptamine hydrochloride (0.067 g, 0.34 mmol) and purified by trituration in ethyl acetate and a minimal amount of cyclohexane, followed by washing with diethyl ether to afford 7f (0.014 g, 12%). C₃₆H₃₀BrN₃O₅.

¹H NMR (400 MHz, CDCl₃) δ 8.20 (s, 1H), 7.68 (d, J=7.7 Hz, 1H), 7.57 (t, J=8.4 Hz, 1H), 7.54-7.44 (m, 5H), 7.32-7.15 (m, 6H), 7.12-7.06 (m, 2H), 7.06-7.01 (m, 1H), 6.89 (s, 1H), 6.85 (d, J=8.2 Hz, 1H), 6.80 (s, 1H), 5.99-5.91 (m, 1H), 5.19 (s, 2H), 4.72 (dd, J=13.5, 7.7 Hz, 1H), 3.53 (dd, J=11.8, 5.9 Hz, 2H), 3.20 (dd, J=13.4, 5.6 Hz, 1H), 3.06 (dd, J=13.3, 8.4 Hz, 1H), 2.88 (dt, J=13.1, 6.4 Hz, 1H), 2.84-2.73 (m, 1H).

¹³C NMR (101 MHz, CDCl₃) δ 177.67, 170.09, 159.03, 158.55, 157.39, 152.34, 136.50, 136.16, 135.38, 134.72, 131.87, 129.44, 128.91, 128.45, 127.44, 127.17, 122.32, 122.19, 121.84, 119.59, 118.58, 115.56, 113.88, 112.34, 111.42, 110.84, 109.06, 70.35, 60.54, 55.24, 39.94, 38.96, 29.82, 25.00, 21.18, 14.33. 36 C INSTEAD OF 32.

HRMS (ESI/QTOF):
calculated for C₃₆H₃₁BrN₃O₅ (M+H⁺): 664.1447,
found: 664.1432.

### EXAMPLE 27

### Preparation of (R)—N-(1-((2-(1H-indol-3-yl)ethyl)amino)-3-(1H-indol-3-yl)-1-oxopropan-2-yl)-5-((4-bromobenzyl)oxy)-4-oxo-4H-chromene-2-carboxamide (7 g)

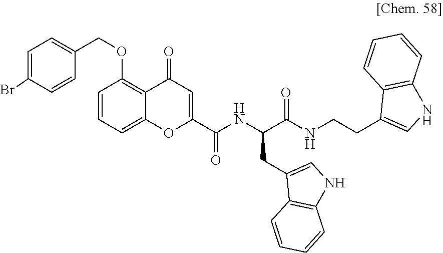 [SMILES: O=C(N[C@H](Cc1c[nH]c2ccccc12)C(=O)NCCc1c[nH]c2ccccc12)c1cc(=O)c2c(OCc3ccc(Br)cc3)cccc2o1]

The crude was prepared according to general procedure F starting from 6e (0.069 g, 0.13 mmol), tryptamine hydrochloride (0.048 g, 0.25 mmol) and purified by trituration in diethyl ether to give 7 g (0.051 mg, 59%). C₃₈H₃₁BrN₄O₅.

¹H NMR (400 MHz, DMSO) δ 10.83 (s, 2H), 8.98 (d, J=8.2 Hz, 1H), 8.34 (t, J=5.3 Hz, 1H), 7.78 (t, J=8.4 Hz, 1H), 7.70 (d, J=7.7 Hz, 1H), 7.60 (q, J=8.6 Hz, 1H), 7.55 (d, J=7.8 Hz, 1H), 7.36-7.26 (m, 3H), 7.18 (dd, J=31.1, 0.9 Hz, 2H), 7.11 (d, J=8.4 Hz, 1H), 7.08-7.01 (m, 2H), 7.00-6.92 (m, 2H), 6.61 (s, 1H), 5.24 (s, 2H), 4.72-4.63 (m, 1H), 3.30-3.14 (m, 2H), 2.82 (t, J=7.3 Hz, 2H). Two signals below the water peak.

¹³C NMR (101 MHz, DMSO) δ 176.46, 170.44, 158.90, 157.64, 156.94, 153.16, 136.27, 136.19, 136.04, 134.99, 131.20, 128.90, 127.24, 127.15, 123.76, 122.67, 120.89, 120.88, 120.58, 118.53, 118.21, 114.43, 112.31, 111.66, 111.33, 110.67, 110.08, 108.95, 69.17, 54.44, 27.38, 25.02. 32C+2 EQ 4C MISSING.

HRMS (ESI/QTOF)
calculated for C₃₈H₃₁BrN₄O₅ (M+H⁺): 703.1556,
found: 703.1553.

### EXAMPLES 28 TO 36

Series 3

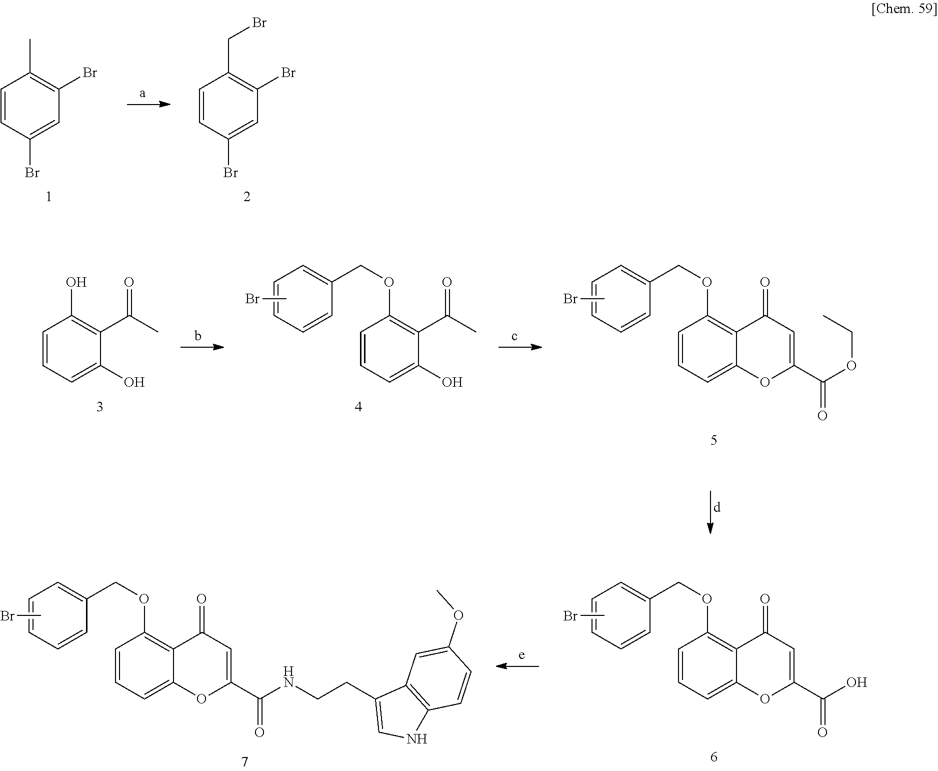

Note: In the following protocols, “molecule number+a” refers to when the bromines are in the 2 and 4 position of the aromatic ring while “molecule number+b” refers to when the bromines are in the 3 and 5 position of the aromatic ring.

General Operating Mode A

2,5-dihydroxyacetophenone 3 (1 equiv) was solubilised in acetone (11 mL/mmol). Then K₂CO₃ (3 equiv) and tetra-n-butylammonium bromide (TBAB) (1.5 equiv) were mixed together, weighed and added to the solution. The resulting suspension was refluxed for 30 min and a solution of dibromo-1-(bromomethyl)benzene (1 equiv) in acetone (4 mL/mmol) was added. The suspension was refluxed for 30 min and then concentrated under vacuum. The reaction was monitored by TLC (cyclohexane/ethyl acetate 7:3).

The reaction mixture was poured into ethyl acetate and acidified water (1M HCl). The aqueous layer was extracted (3 times) with ethyl acetate, then the combined organic layers were washed (1 time) with acidified water (1M HCl) and brine. The combined organic layers were dried over MgSO₄, filtered and evaporated under vacuum. Finally, the crude was dried under high vacuum.

General Procedure B

Sodium (6 equiv) was solubilised in cold anhydrous ethanol (3 mL/mmol) to give a fresh solution of sodium ethanoate. This solution was poured dropwise into a cold (0° C.) solution of 4 (1 equiv) in dry THF (same volume as ethanol). Then diethyl oxalate (4 equiv) was added to the solution and stirred at room temperature for 30 min. The resulting solution was warmed to 50° C. and monitored by TLC (cyclohexane/ethyl acetate 3:2). Precipitation of the reaction intermediate occurred during the reaction.

After 4 hours, drops of 37% HCl were added to the solution until the precipitate became white. The reaction was refluxed for 1.5 hours after the colour change. Then the reaction mixture was evaporated and poured into ethyl acetate and acidified water (1M HCl). The aqueous layer was extracted (3 times) with ethyl acetate until the colour changed. The combined organic layers were washed (once) with acidified water (1M HCl) and brine and dried over MgSO₄ before being evaporated.

General Operating Procedure C

A solution of K₂ CO₃ (1.3 equiv) in water (15 mL/mmol) was added to a solution of 5 (1 equiv) in THF (30 mL/mmol) and ethanol (EtOH) (10 mL/mmol). The resulting solution was heated to 50° C. and stirred for 1.5 hours. The reaction was monitored by TLC (cyclohexane/ethyl acetate 7:3). The reaction mixture was concentrated and poured into dichloromethane and acidified water (1M HCl).

To increase the solubility of the desired product in the organic layer, a few drops of methanol were added. The aqueous layer was extracted (3 times) with dichloromethane and the combined organic layers were washed (once) with acidified water (1M HCl) and brine. Then the combined organic layers were dried over MgSO₄, filtered and evaporated.

### EXAMPLE 28

### Preparation of 2,4-dibromo-1-(bromomethyl)benzene (2)

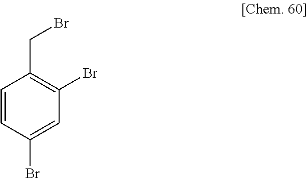

2,4-dibromotoluene 1 (1000 g, 4.00 mmol) and freshly purified N-bromosuccinimide (NBS) (0.925 g, 5.20 mmol) were solubilised in 12 mL of 1,2-dichloroethane under an inert atmosphere. The solution was refluxed for 10 min and azobisisobutyronitrile (AIBN) (0.328 g, 2.00 mmol) was added. The resulting suspension was stirred and refluxed for 6 hours. The reaction was monitored by TLC (100% cyclohexane). Then the reaction mixture was evaporated and a cold 1:1 cyclohexane/dichloromethane solution was added to precipitate side products (white solid). After filtration and evaporation, crude product 2 (1.476 g, 4.49 mmol) was used directly without purification. C₇H₅Br₃

### EXAMPLE 29

### Preparation of 1-(2-((2, 4-dibromobenzyl)oxy)-6-hydroxyphenyl)ethan-1-one (4a)

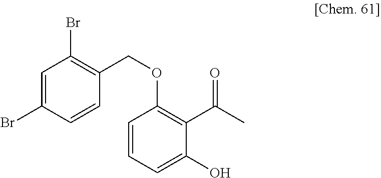 [SMILES: CC(=O)c1c(O)cccc1OCc1ccc(Br)cc1Br]

The crude was prepared according to general procedure A starting from 2 (1.315 g, 4.00 mmol) and 3 (1.000 g, 4.00 mmol). The crude was precipitated with a 1:1 cyclohexane/dichloromethane solution and then recrystallised in isopropanol to afford 4a (0.793 g, 50%). C₁₅H₁₂Br₂O₃.

¹H NMR (400 MHz, DMSO) δ 11.64 (s, 1H), 7.95 (d, J=1.8 Hz, 1H), 7.66 (dd, J=8.2, 1.8 Hz, 1H), 7.54 (d, J=8.2 Hz, 1H), 7.32 (t, J=8.3 Hz, 1H), 6.62 (d, J=8.3 Hz, 1H), 6.55 (d, J=8.2 Hz, 1H), 5.13 (s, 2H), 2.45 (s, 3H).

¹³C NMR (101 MHz, DMSO) δ 203.27, 159.54, 157.71, 134.92, 134.60, 133.83, 132.06, 131.01, 123.92, 122.19, 114.68, 109.93, 103.16, 69.38, 32.88

HRMS (ESI/LTQ Orbitrap):
calculated for C₁₅H₁₁O₃Br₂ (M−H⁺): 396.9080,
found: 396.9082.

### EXAMPLE 30

### Preparation of 1-(2-((3,5-dibromobenzyl)oxy)-6-hydroxyphenyl)ethan-1-one (4b)

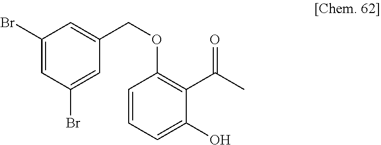 [SMILES: CC(=O)c1c(O)cccc1OCc1cc(Br)cc(Br)c1]

The crude was prepared according to general procedure A starting from 1,3-dibromo-5-(bromomethyl)benzene (1.315 g, 4.00 mmol) and 3 (1.000 g, 4.00 mmol). It was purified by trituration in diethyl ether to afford 4b (1.103 g, 70%). C₁₅H₁₂O₃Br₂.

¹H NMR (400 MHz, DMSO) δ 11.53 (s, 1H), 7.79 (s, 1H), 7.69 (d, J=1.5 Hz, 2H), 7.29 (t, J=8.3 Hz, 1H), 6.58 (d, J=8.3 Hz, 1H), 6.54 (d, J=8.2 Hz, 1H), 5.17 (s, 2H), 2.53-2.46 (m, 5H) 13C NMR (101 MHz, DMSO) δ 203.16, 158.98, 157.30, 141.43, 133.41, 132.81, 129.45, 122.49, 115.30, 109.78, 103.40, 68.24, 32.91

HRMS (ESI/LTQ Orbitrap):
calculated for C₁₅H₁₁O₃Br₂ (M−H^{m}) 396.9080,
found: 396.9078.

### EXAMPLE 31

### Preparation of ethyl 5-((2,4-dibromobenzyl)oxy)-4-oxo-4H-chromene-2-carboxylate (5a)

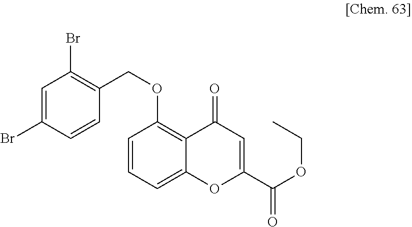 [SMILES: CCOC(=O)c1cc(=O)c2c(OCc3ccc(Br)cc3Br)cccc2o1]

The crude was prepared according to general procedure B starting from 4a (0.435 g, 1.09 mmol) and diethyl oxalate (0.636 g, 4.35 mmol). The resulting oil was solidified under high vacuum and isopropanol was added and heated.

The resulting suspension was filtered, and the paste-like product was dissolved in dichloromethane to obtain a white solid after evaporation. The desired product 5a (0.144 g, 27%). C₁₉H₁₄O₅Br₂.

¹H NMR (400 MHz, DMSO) δ 8.08 (d, J=8.3 Hz, 1H), 7.94 (d, J=1.9 Hz, 1H), 7.80 (t, J=8.4 Hz, 1H), 7.74 (dd, J=8.3, 1.9 Hz, 1H), 7.29 (d, J=8.5 Hz, 1H), 7.15 (d, J=8.3 Hz, 1H), 6.81 (s, 1H), 5.19 (s, 2H), 4.40 (q, J=7.1 Hz, 2H), 1.36 (t, J=7.1 Hz, 3H)

¹³C NMR (101 MHz, DMSO) δ 176.44, 159.99, 157.23, 157.22, 150.30, 135.46, 135.43, 134.04, 130.85, 130.60, 121.82, 121.29, 115.42, 114.59, 110.90, 108.90, 69.29, 62.62, 13.87

HRMS (ESI/LTQ Orbitrap):
calculated for C₁₉H₁₅O₅Br₂ (M+H⁺): 480.9281,
found: 480.9271.

### EXAMPLE 32

### Preparation of ethyl 5-((3,5-dibromobenzyl)oxy)-4-oxo-4H-chromene-2-carboxylate (5b)

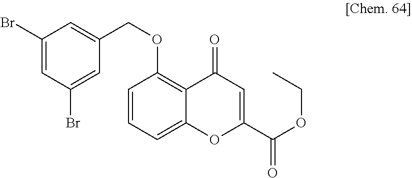 [SMILES: CCOC(=O)c1cc(=O)c2c(OCc3cc(Br)cc(Br)c3)cccc2o1]

The crude was prepared according to general procedure B starting from 4b (1.103 g, 2.76 mmol) and diethyl oxalate (1.612 g, 14.03 mmol). It was purified by trituration in diethyl ether to afford 5b (0.785 g, 59%). C₁₉H₁₄Br₂O₅.

¹H NMR (400 MHz, DMSO) δ 7.91 (d, J=1.6 Hz, 2H), 7.81-7.74 (m, 2H), 7.26 (d, J=8.1 Hz, 1H), 7.08 (d, J=8.3 Hz, 1H), 6.83 (s, 1H), 5.27 (s, 2H), 4.38 (q, J=7.1 Hz, 2H), 1.34 (t, J=7.1 Hz, 3H).

¹³C NMR (101 MHz, DMSO) δ 176.55, 159.99, 157.32, 157.22, 150.28, 141.68, 135.40, 132.26, 128.38, 122.44, 115.46, 114.62, 110.82, 108.83, 68.21, 62.60, 13.86

HRMS (ESI/LTQ Orbitrap):
calculated for C₁₉H₁₅Br₂O (M+H⁺): 480.9281,
found: 480.9274.

### EXAMPLE 33

### Preparation of 5-((2,4-dibromobenzyl)oxy)-4-oxo-4H-chromene-2-carboxylic acid (6a)

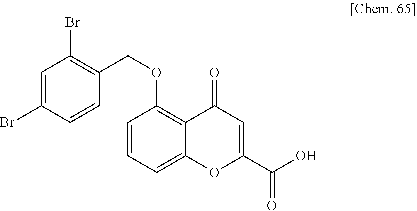 [SMILES: O=C(O)c1cc(=O)c2c(OCc3ccc(Br)cc3Br)cccc2o1]

The crude was prepared according to general procedure C starting from 5a (0.144 g, 0.30 mmol) and purified by trituration in 1:1 diethyl ether/dichloromethane to afford 6a (0.074 g, 55%). C₁₇H₁₀Br₂O₅.

¹H NMR (400 MHz, DMSO) δ 8.07 (d, J=8.3 Hz, 1H), 7.90 (d, J=1.9 Hz, 1H), 7.76 (t, J=8.4 Hz, 1H), 7.70 (dd, J=8.3, 1.9 Hz, 1H), 7.24 (d, J=8.3 Hz, 1H), 7.10 (d, J=8.3 Hz, 1H), 6.75 (s, 1H), 5.15 (s, 2H).

¹³C NMR (101 MHz, DMSO) δ 176.72, 161.42, 157.30, 157.17, 151.21, 135.46, 135.28, 133.97, 130.81, 130.55, 121.71, 121.23, 115.20, 114.54, 110.90, 108.69, 69.23, 64.89

HRMS (ESI/LTQ Orbitrap):
calculated for C₁₇H₁₁Br₂O₅ (M+H⁺): 454.8948,
found: 454.8937.

### EXAMPLE 34

### Preparation of 5-((3,5-dibromobenzyl)oxy)-4-oxo-4H-chromene-2-carboxylic Acid (6b)

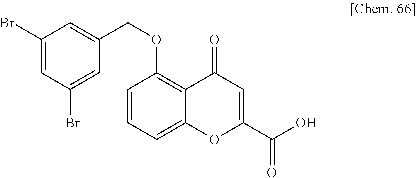 [SMILES: O=C(O)c1cc(=O)c2c(OCc3cc(Br)cc(Br)c3)cccc2o1]

The crude was prepared according to general procedure C starting from 5b (0.785 g, 1.63 mmol) and purified by trituration in 1:1 diethyl ether/dichloromethane to afford 6b (0.493 g, 67%). C₁₇H₁₀Br₂O₅.

¹H NMR (400 MHz, DMSO) δ 7.91 (s, 2H), 7.82-7.72 (m, 2H), 7.24 (d, J=8.4 Hz, 1H), 7.06 (d, J=8.3 Hz, 1H), 6.79 (s, 1H), 5.27 (s, 2H)

¹³C NMR (101 MHz, DMSO) δ 176.82, 161.44, 157.31, 157.29, 151.21, 141.70, 135.23, 132.22, 128.34, 122.42, 115.23, 114.58, 110.83, 108.67, 68.19

HRMS (ESI/LTQ Orbitrap):
calculated for C₁₇H₁₁Br₂O₅ (M+H⁺): 454.8948,
found: 454.8941.

### EXAMPLE 35

### Preparation of 5-((2,4-dibromobenzyl)oxy)-N-(2-(5-methoxy-1H-indol-3-yl)ethyl)-4-oxo-4H-chromene-2-carboxamide (7a)

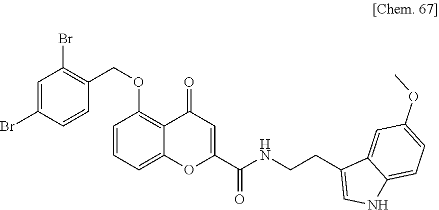 [SMILES: COc1ccc2[nH]cc(CCNC(=O)c3cc(=O)c4c(OCc5ccc(Br)cc5Br)cccc4o3)c2c1]

6a (0.074 g, 0.16 mmol) was solubilised in 2 mL of anhydrous DMF. Subsequently, DIEA (0.084 g, 0.65 mmol) and TBTU (0.105 g, 0.33 mmol) were added successively to the solution. After complete dissolution, 5-methoxytryptamine (0.074 g, 0.33 mmol) was added and the resulting solution was stirred at room temperature for 24 hours. The reaction was monitored by TLC (cyclohexane/ethyl acetate 1:4).

The reaction mixture was poured into acidified water (1M HCl) and extracted (3 times) with dichloromethane. The organic phases were collected and washed (once) with acidified water (1M HCl), basified water (10% NaOH) and brine before being dried over MgSO₄, filtered and evaporated. The resulting oil was precipitated with a few drops of diethyl ether.

After filtration, the crude product was purified using a 12 g silica column and as eluent cyclohexane/dichloromethane 4:1 to 100% dichloromethane. After elution of a first product, the desired product was obtained with 9:1 dichloromethane/methanol. The desired product 7a (0.006 g, 5.6%) is a solid. C₂₈H₂₂Br₂N₂O₅

¹H NMR (500 MHz, CDCl₃) δ 8.12 (d, J=8.4 Hz, 1H), 8.00 (s, 1H), 7.72 (d, J=1.9 Hz, 1H), 7.60-7.55 (m, 2H), 7.31 (d, J=8.8 Hz, 1H), 7.10 (dd, J=9.8, 2.3 Hz, 2H), 7.05 (s, 1H), 6.95-6.89 (m, 3H), 6.85 (dd, J=8.4, 0.5 Hz, 1H), 5.16 (s, 2H), 3.84-3.78 (m, 5H), 3.11 (t, J=6.6 Hz, 2H)

¹³C NMR (126 MHz, CDCl₃) δ 177.59, 159.14, 158.11, 157.22, 154.32, 152.90, 134.78, 134.48, 134.36, 131.54, 131.28, 130.13, 127.80, 122.96, 121.80, 121.15, 115.29, 113.68, 112.63, 112.36, 112.17, 110.59, 108.41, 100.40, 69.86, 55.84, 40.52, 24.94

HRMS (ESI/LTQ Orbitrap)
calculated for C₂₈H₂₃Br₂N₂O₅ (M+H⁺): 626.9948,
found: 626.9929.

### EXAMPLE 36

### Preparation of ((3,5-dibromobenzyl)oxy)-N-(2-(5-methoxy-1H-indol-3-yl)ethyl)-4-oxo-4H-chromene-2-carboxamide (7b)

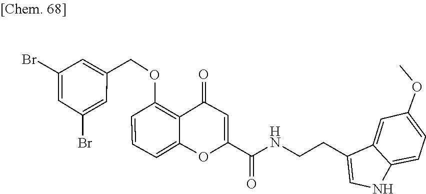

6b (0.100 g, 0.22 mmol) was solubilised in anhydrous DMF (10 mL) and stirred until completely dissolved. Then (1H-benzotriazol-1-yloxy) (tri-1-pyrrolidinyl)phosphonium hexafluorophosphate (PyBOP) coupling agent (0.195 g, 0.44 mmol) was added to the solution followed by DIEA (0.114 g, 0.88 mmol). The solution was stirred at room temperature for 1 hour. A colour change appeared.

Then 5-methoxytryptamine (0.100 g, 0.44 mmol) was added and the solution was stirred at room temperature for 2 days. With no change in cyclohexane/ethyl acetate 1:1 TLC, bis(2-oxo-1,3-oxazolidin-3-yl)phosphinic chloride (BOP—Cl) (0.112 g, 0.44 mmol) was added to the solution. After 2 days, a 1:1 cyclohexane/ethyl acetate TLC showed the formation of products.

The reaction mixture was evaporated and poured into ethyl acetate. The organic phase was washed (3 times) with basified water (saturated K₂CO₃), then acidified water (1M HCl) and brine. The organic layer was dried over MgSO₄ and evaporated to obtain a brown solid.

The solid was purified by silica column chromatography using a dry sample and an eluent such as 100% dichloromethane followed by 2.4:0.1 dichloromethane/methanol. The 5 mL fractions were kept overnight in a fume hood to precipitate the desired product. After filtration, the desired product 7b, (0.007 g, 6.6%) was obtained. C₂₈H₂₂O₅N₂Br₂

¹H NMR (400 MHz, DMSO) δ 10.71 (s, 1H), 9.23 (t, J=5.8 Hz, 1H), 7.94 (d, J=1.3 Hz, 2H), 7.85-7.79 (m, 2H), 7.30 (d, J=8.4 Hz, 1H), 7.24 (d, J=8.7 Hz, 1H), 7.19 (d, J=2.1 Hz, 1H), 7.12-7.06 (m, 2H), 6.76-6.70 (m, 2H), 5.30 (s, 2H), 3.75 (s, 3H), 3.58 (dd, J=14.2, 6.6 Hz, 2H), 2.97 (t, J=7.4 Hz, 2H)

¹³C NMR (126 MHz, DMSO) δ 177.14, 159.37, 157.83, 157.49, 154.21, 153.51, 142.26, 135.54, 132.78, 131.88, 128.94, 128.03, 123.91, 122.94, 114.95, 112.57, 112.53, 111.72, 111.56, 111.36, 109.32, 100.65, 68.76, 55.81, 25.35

HRMS (ESI/LTQ Orbitrap):
calculated for C₂₈H₂₃O₅N₂Br₂ (M+H⁺): 626.9948,
found: 626.9937.

### EXAMPLE 37

### Biological Evaluation

### Materials

High glucose DMEM (Dulbecco/Vogt modified Eagle's minimal medium) with GlutaMAX™ (Gibco) and fetal calf serum (FBS, GE Healthcare Hyclone) were purchased from Fisher Scientific. Penicillin/streptomycin (10,000 U/10 mg per ml), G418, trypsin and Dulbecco's phosphate buffered saline (DPBS) were purchased from Sigma Aldrich (France), as well as mitoxantrone (MX), rhodamine 123 (R123), calcein-AM (cAM) and 3-(4,5-dimethylthiazol-2-yl)-2,5-diphenyltetrazolium bromide (MTT). All commercial products were of the highest purity available.

### Compounds

All chromone derivatives were dissolved in dimethyl sulphoxide (DMSO) and then diluted in high glucose DMEM. Stock solutions were stored at −20° C. and warmed to 25° C. just prior to use.

### Cell Lines and Cultures

ABCB1-transfected NIH/3T3, ABCC1-transfected Flp-In 293 and ABCG2-transfected HEK293 cells and their empty plasmid counterparts were generated as previously described (Borst, P.; Elferink, R. O. Mammalian ABC Transporters in Health and Disease. Annu. Rev. Biochem. 2002, 71 (1), 537-592).

Specifically, the ABCG2-transfected HEK293 monoclonal cell line was selected after fluorescence-activated cell sorting (FACS) using a phycoerythrin-coupled 5D3 antibody (Santa Cruz Biotech) as an endogenous expression reporter.

Cells were grown and maintained in high glucose DMEM with GlutaMAX™ supplemented with 10% heat-inactivated fetal calf serum (FBS) and 1% penicillin/streptomycin in a humidified atmosphere at 37° C. with 5% CO₂. In addition, 200 μg/mL hygromycin B, 90 ng/mL colchicine or 750 μg/mL G418 were added to the growth medium as selection agents for NIH/3T3, Flp-In 293 or HEK293 transfected cells, respectively.

### Cytotoxicity Tests

The cytotoxicity of compounds was determined using a colorimetric MTT assay as reported in the literature (Linton, K. J. Structure and Function of ABC Transporters. Physiology 2007, 22 (2), 122-130. Sharom, F. J. ABC Multidrug Transporters: Structure, Function and Role in Chemoresistance. Pharmacogenomics 2008, 9 (1), 105-127).

Briefly, cells were seeded in 96-well plates at a density of 1×10⁵ cells/well for a total growth medium volume of 100 μL and incubated overnight. Then, 100 μL of fresh medium containing increasing concentrations of compounds (dissolved in DMSO in a concentration range of 0, 2 and 20 μM) to be tested were added to each well while the DMSO control was fixed at 0.5% (v/v). After incubation for 72 hours, 22 μL of MTT dye in PBS (5 mg/mL) was added to each well and the plates were incubated for a further 4 hours at 37° C. After removal of the medium and drying, the formazan dye crystals were solubilised with 200 μL of DMSO/ethanol (1:1, v/v). The absorbance was measured using spectrophotometry at 570 nm and 690 nm as reference wavelength. The effect of each compound on cell viability in all cell lines was calculated as the difference in absorbance between the test and control media wells.

The cytotoxicity results of the compounds according to the invention are shown in Table 1.

| | Cell viability(%) | | | |
|---|---|---|---|---|
| Compound | [Compound] 1 pM | | [Compound] 10 pM | |
| 5a (series 1) | / | | / | |
| 5b (series 1) | 78 ± | 0 | 87 ± | 1 |
| 5c (series 1) | 82 ± | 2 | 76 ± | 1 |
| 5d (series 1) | 75 ± | 1 | 82 ± | 6 |
| 5th (series | 81 ± | 3 | 88 ± | 1 |
| 1) | | | | |
| 5f (series 1) | 82 ± | 6 | 84 ± | 3 |
| 5g (series 1) | 73 ± | 4 | 77 ± | 0 |
| 5h (series 1) | 83 ± | 2 | 102 ± | 1 |
| 5i (series 1) | 78 ± | 0 | 87 ± | 1 |
| 7a (series 2) | 104 ± | 16 | 102 ± | 12 |
| 7b (series 2) | 100 ± | 18 | 104 ± | 11 |
| 7c (series 2) | 106 ± | 30 | 89 ± | 22 |
| 7d (series 2) | 118 ± | 12 | 101 ± | 12 |

| | | |
|---|---|---|
| DMSO 0.5 % | 100 | 100 |

The compounds according to the invention therefore exhibit very low cytotoxicity or no cytotoxicity.

MDR-Related Drug Efflux Inhibition Tests

The cells were seeded in 96-well plates at a density of 5×10⁴ cells/well in 200 μL of medium and incubated overnight. Then the growth medium was changed to fresh medium containing the compounds and in the presence of 4 μM MX as a fluorescent probe for BCRP-mediated efflux at a final concentration of 0.5% (v/v) DMSO. After 30 min incubation at 37° C., the medium was removed, and the cells were washed with 100 μL of Dulbecco's phosphate-buffered saline (DPBS) followed by dissociation of the cells for 5 min at 37° C. mediated by 25 μL trypsin. Finally, trypsin was neutralised with 175 μL of DPBS ice-cold with 2% bovine serum albumin (BSA) and the cells were carefully resuspended. As a selectivity assay, the same experiment was performed for P-gp and MRP1-mediated efflux with 0.5 μM R123 or 0.2 μM cAM as respective fluorescent substrates instead of MX.

Intracellular fluorescence was measured with a MacsQUANT VRB Analyzer flow cytometer (Miltenyi Biotec) with at least 5000 events recorded. While MX was excited at 635 nm and the fluorescence emission recorded in a 655-730 nm window, R123 and cAM were excited at 488 nm and recorded in a 525/50 nm filter. The compound inhibition yield was estimated by the following equation:

$$\% of inhibition = 100 * \frac{[ ( G {2}_{FA} - G {2}_{F B G} ) - ( G {2}_{S} - G {2}_{F B G} ) ]}{[ ( H E {K}_{F A} - H E {K}_{F B G} ) - ( G {2}_{S} - G {2}_{F B G} ) ]} \qquad [ Math . 1 ]$$

where G2_{FA} is the fluorescence emission (a.u.) of accumulated fluorophore in cells expressing the efflux pump incubated with a fluorescent substrate and the test compound. G2_{FBG} is the resulting background fluorescence emission (a.u.) in cells transfected with ABCG2 (no substrate or test compound). G2_{S} is the fluorescence emission (a.u.) of accumulated fluorophore in cells expressing the efflux pump incubated with substrate only. HEK_{FA} is the fluorescence emission (a.u.) of accumulated fluorophore in control cells incubated with the substrate and test compound. HEK_{FBG} is the resulting background fluorescence emission (a.u.) in control cells (no substrate or test compound). All values are given as the geometric mean fluorescence emission (a.u.) in a 655-730 nm filter (635 nm excitation) measured over 5000 events. The tests were performed in triplicate.

### Activity of Chromones as ABCG2 Inhibitors

TABLE 2

| | | |
|---|---|---|
| Absolute | Inhibition (%) | IC₅₀ |

| | | | | | |
|---|---|---|---|---|---|
| Input | Position of R₁ Br | configuration | 1 μM | 10 μM | (μM) |

SERIES 1

| | | | | | | |
|---|---|---|---|---|---|---|
| 5a | 2 | —CH(CH₃)CH₂CH₃ | S | 78 ± 9 | 143 ± 22 | 0.10 ± 0.01 |
| 5b | 4 | —CH(CH₃)CH₂CH₃ | S | — | 115 ± 14% | 0.59 ± 0.08 |
| 5c | 2 | —CH₂CH(CH₃)₂ | S | 115 ± 17 | 101.5 ± 21.7 | 0.14 ± 0.04 |
| 5d | 2 | —CH(CH₃)₂ | S | 87 ± 7 | 148.2 ± 6.7 | 0.05 ± 0.03 |
| 5e | 2 | —CH₂Ph | S | 100 ± 14 | 84.6 ± 2.0 | 0.10 ± 0.07 |
| 5g | 2 | —CH₂(3-indolyl) | S | 93.7 ± 17 | 39.7 ± 3.7 | n.a. |
| 5f | 4 | —CH₂Ph | S | 92 ± 15 | 114 ± 11 | 0.27 ± 0.11 |
| 5h | 4 | —CH₂(3-indolyl) | S | 92 ± 15 | 87.7 ± 12 | 0.48 ± 0.07 |
| 5i | 4 | —CH₂(3-indolyl) | R | 85 ± 12 | 96.3 ± 19 | 0.29 ± 0.05 |
| 6a | 2 | —CH(CH₃)CH₂CH₃ | S | 0.0 ± 0.7 | 1.6 ± 3.1 | n.a. |
| 6d | 4 | —CH₂Ph | S | 4.0 ± 0.7 | 6.5 ± 0.0 | n.a. |
| 6e | 4 | —CH₂(3-indolyl) | R | 6.1 ± 0.6 | 11.2 ± 2.0 | n.a. |

SERIES 2

| | | | | |
|---|---|---|---|---|
| 7a | 2 | —CH(CH₃)CH₂CH₃ | S | 0.07 ± 0.01 |
| 7b | 2 | —CH(CH₃)CH₂CH₃ | S | 0.07 ± 0.01 |
| 7c | 4 | —CH(CH₃)CH₂CH₃ | S | 0.25 ± 0.10 |
| 7d | 4 | —CH(CH₃)CH₂CH₃ | S | 0.11 ± 0.03 |

SERIES 3

| | | | | | |
|---|---|---|---|---|---|
| 7a | 2, 4 | H | / | / | 0.05 ± 0.01 |
| 7b | 3, 5 | H | / | / | 0.10 ± 0.01 |
| MBL- | 4 | | | | 0.13 ± 0.09 |
| II-141 | | | | | 0.09 |
| Ko143 | | | | | |

The above table shows that the compounds show good IC50 values. In particular, compounds 5d in series 1, 7a and 7b in series 2 and 7a in series III show better results than the MBL-II-141 inhibitor and the reference inhibitor Ko143.
Chromone Selectivity for BCRP Vs. P-Gp and MRP1

| | | P-gp | MRP1 | BCRP |
|---|---|---|---|---|
| entry | [Inhibitor] | | | |
| | μM | Inhibition (%) | | |
| 5a (series | 1 | | | / |
| 1) | 10 | | | |
| 5c (series | 1 | 7.0 ± 1.1 | 20.2 ± 2.7 | 114.5 |
| 1) | 10 | 4.8 ± 0.7 | 19.2 ± 2.2 | / |
| 5d (series | 1 | 7.9 ± 0.7 | 12.6 ± 1.3 | 86.6 |
| 1) | 10 | 6.5 ± 1.9 | 17.8 ± 0.9 | / |
| 5th (series | 1 | 4.9 ± 0.8 | 25.8 ± 0.8 | 100.2 |
| 1) | 10 | 5.7 ± 1.4 | 29.2 ± 1.5 | / |
| 5f (series | 1 | 6.4 ± 1.5 | 23.0 | 91.6 |
| 1) | 10 | 6.8 ± 1.4 | 19.0 | / |
| 5g (series | 1 | 7.4 ± 0.3 | 23.0 ± 2.5 | 93.7 |
| 1) | 10 | 5.3 ± 0.5 | 16.0 ± 2.5 | / |
| 5h (series | 1 | 7.4 ± 2.5 | 10.3 | 96.4 |
| 1) | 10 | 6.6 ± 1.4 | 17.4 | / |
| 5i (series | 1 | 7.2 ± 1.3 | 11.9 | 84.8 |
| 1) | 10 | 6.1 ± 1.1 | 12.6 | / |
| 6a (series | 1 | 7.8 1.8 | 17.1 | / |
| 1) | 10 | 7.9 ± 2.3 | 13.1 ± 2.4 | / |
| 6b (series | 1 | 7.7 ± 1.9 | 21.9 ± 4.6 | / |
| 1) | 10 | 7.1 ± 0.6 | 20.6 ± 1.2 | / |
| 6c (series | 1 | 7.7 ± 2.0 | 20.0 ± 3.7 | / |
| 1) | 10 | 6.3 ± 1.4 | 18.1 ± 1.2 | / |
| 7a (series | 1 | 0.1 ± 0.0 | 11.1 ± 0.8 | 84.8 ± 7.4 |
| 2) | 10 | 0.2 ± 0.0 | 10.8 ± 2.8 | / |
| 7b (series | 1 | 1.7 ± 2.4 | 10.3 ± 0.7 | 82.5 ± 9.7 |
| 2) | 10 | 2.4 ± 0.5 | ± 0.6 | / |
| 7c (series | 1 | 2.4 ± 2.1 | 11.5 ± 0.4 | 72.4 ± 7.1 |
| 2) | 10 | 10.6 ± 0.3 | 15.3 ± 1.5 | / |
| 7d (series | 1 | 3.6 ± 0.2 | 10.9 ± 1.4 | 52.3 ± 5.1 |
| 2) | 10 | 5.9 ± 0.1 | 14.7 ± 1.0 | |

1. A compound of formula (I): 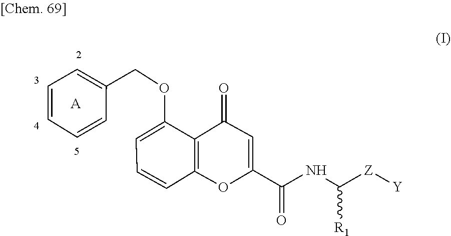
or pharmaceutically acceptable enantiomer, salt or solvate thereof, or a mixture thereof,
in which:
the ring A is unsubstituted or substituted in the 2, 3, 4, 5 position by one or two of F; Cl; Br; I; OR, with R Me, Et, Pr, i-Pr, n-Bu; O—CH₂—(O—CH₂CH₂)ₙ—O—CH₃, with n 3, 4, 5, 6,
Z is 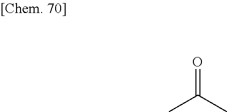
or —CH₂—,
Y═—OH; —OMe;-OEt; —OPr; —NH₂; —NHMe; —N(Me)₂; —N(Me)OCH₃; —NH—(CH₂)₂-(3-indolyl); —NH(CH₂)₂-3-((5-hydroxy)indolyl); —NH—CH(R₃)—COR₂, with R₂ selected from:
OH; —OMe;-OEt; —OPr; —NH₂; —NHMe; —N(Me)₂; —N(Me)OCH₃; 3-(5-methoxy)indolyl; —NH—(CH₂)₂-(3-indolyl); —NH(CH₂)₂-3-((5-hydroxy)indolyl); —NH(CH₂)₂-3-((5-methoxy)indolyl) 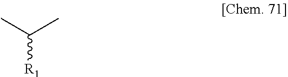
in formula (I) and R₃ of the substituent —NH—CH(R₃)—COR₂ of Y are independently selected from: H or 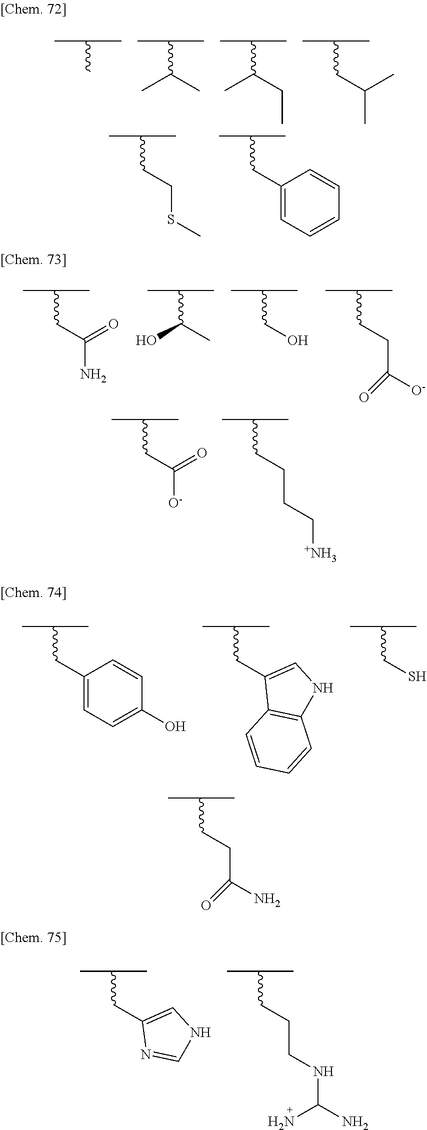
with the exception of compounds with simultaneous Br in the 4-position of ring A, R₁═CH(CH₃)₂ or CH₂CH(CH₃)₂ or CH(CH₃)CH₂CH₃, Z= 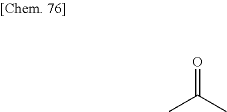
and Y═—OH or —OMe.

2. The compound according to claim 1, wherein the ring A is substituted in position 2, 3, 4, 5 by one or two Br and Y═—OH; —OMe; —NH—(CH₂)₂-(3-indolyl); —NH(CH₂)₂-3-((5-hydroxy)indolyl); —NH—CH(R₃)—COR₂, R₁, R₂ and R₃ being as defined in claim 1.

3. The compound according to claim 1, wherein Y is —NH—(CH₂)₂-(3-indolyl) or —NH(CH₂)₂-3-((5-hydroxy)indolyl) with the proviso that:

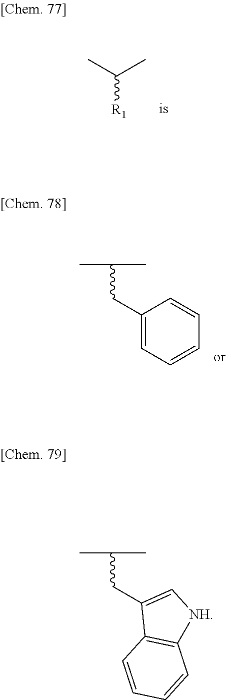

4. The compound according to claim 1 selected from:
methyl (5-((2-bromobenzyl)oxy)-4-oxo-4H-chromene-2-carbonylamino)-L-alloisoleucinate;
methyl (5-((2-bromobenzyl)oxy)-4-oxo-4H-chromene-2-carbonylamino)-L-leucinate;
methyl (5-((2-bromobenzyl)oxy)-4-oxo-4H-chromene-2-carbonylamino)-L-valinate;
methyl (5-((2-bromobenzyl)oxy)-4-oxo-4H-chromene-2-carbonylamino)-L-phenylalaninate;
methyl (5-((4-bromobenzyl)oxy)-4-oxo-4H-chromene-2-carbonylamino)-L-phenylalaninate;
methyl (5-((2-bromobenzyl)oxy)-4-oxo-4H-chromene-2-carbonylamino)-L-tryptophanate;
methyl (5-((4-bromobenzyl)oxy)-4-oxo-4H-chromene-2-carbonylamino)-L-tryptophanate;
methyl (5-((4-bromobenzyl)oxy)-4-oxo-4H-chromene-2-carbonylamino)-D-tryptophanate;
(5-((2-bromobenzyl)oxy)-4-oxo-4H-chromene-2-carbonylamino)-L-alloisoleucine;
(5-((2-bromobenzyl)oxy)-4-oxo-4H-chromene-2-carbonylamino)-L-phenylalanine;
(5-((4-bromobenzyl)oxy)-4-oxo-4H-chromene-2-carbonylamino)-L-phenylalanine;
(5-((4-bromobenzyl)oxy)-4-oxo-4H-chromene-2-carbonylamino)-D-tryptophan;
methyl (5-((2-bromobenzyl)oxy)-4-oxo-4H-chromene-2-carbonylamino)-L-alloisoleucyl-L-valinate;
methyl (5-((2-bromobenzyl)oxy)-4-oxo-4H-chromene-2-carbonylamino)-L-alloisoleucyl-L-leucinate;
methyl (5-((4-bromobenzyl)oxy)-4-oxo-4H-chromene-2-carbonylamino)-L-alloisoleucyl-L-valinate;
methyl (5-((4-bromobenzyl)oxy)-4-oxo-4H-chromene-2-carbonylamino)-L-alloisoleucyl-L-leucinate;
(S)-5-((2-bromobenzyl)oxy)-N-(1-((2-(5-hydroxy-1H-indol-3-yl)ethyl)amino)-1-oxo-3-phenylpropan-2-yl)-4-oxo-4H-chromene-2-carboxamide;
(S)—N-(1-((2-(1H-indol-3-yl)ethyl)amino)-1-oxo-3-phenylpropan-2-yl)-5-((4-bromobenzyl)oxy)-4-oxo-4H-chromene-2-carboxamide; and
(R)—N-(1-((2-(1H-indol-3-yl)ethyl)amino)-3-(1H-indol-3-yl)-1-oxopropan-2-yl)-5-((4-bromobenzyl)oxy)-4-oxo-4H-chromene-2-carboxamide.

5. A process for obtaining the compounds according to claim 1, wherein it comprises the steps:
(a) an alkylating compound of the formula 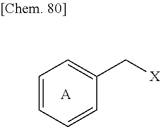
wherein the ring A is as defined in claim 1, and X is a halogen selected from F, Cl, Br and I, is reacted on 2,6-dihydroxyacetophenone of the formula 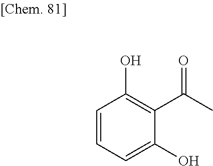
at the reflux temperature of acetone and in acetone to give the intermediate of formula 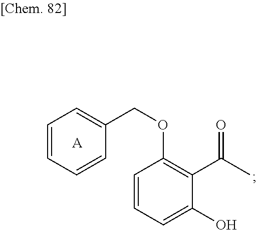
(b) the intermediate obtained in step (a) is reacted with diethyl oxalate of formula 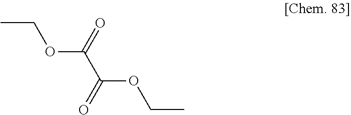
at a temperature of 0° C.-50° C. and in a mixture of tetrahydrofuran (THF)/ethanol (1:1) to give the intermediate of formula 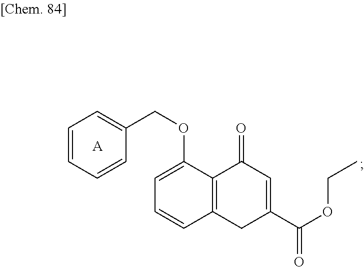
(c) the intermediate obtained in step (b) is reacted by a hydrolysis reaction of the ester function at a temperature of 50° C., in an acidic or basic medium, in a THF/ethanol/water solvent (3:1:1.5) in order to obtain the intermediate of formula 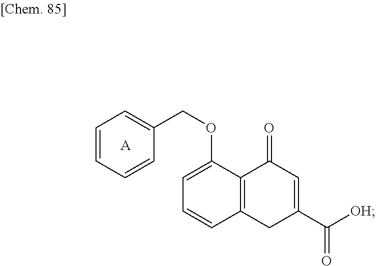
(d) the intermediate obtained in step (c) is reacted with a coupling compound of the formula 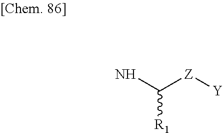
R₁, Z and Y being as defined in claim 1, at room temperature in anhydrous DMF to form an amide bond to give the compound of formula (I).

6. A method for treating breast cancer, the method comprising: administering to a patient in need thereof an effective amount of a compound of formula (I) for inhibition of a multi-drug resistance protein of the breast cancer, the multi-drug resistance protein including Breast Cancer Resistance Protein BCRP/ABCG2, wherein the compound of formula (I) is: 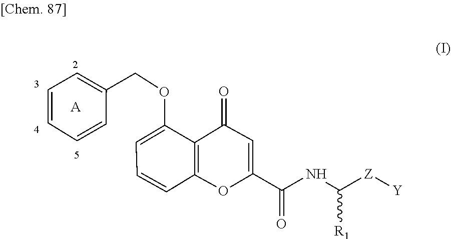
or pharmaceutically acceptable enantiomer, salt or solvate thereof, or a mixture thereof,
in which:
the ring A is unsubstituted or substituted in the 2, 3, 4, 5 position by one or two of F; Cl; Br; I; OR, with R=Me, Et, Pr, i-Pr, n-Bu; O—CH₂—(O—CH₂CH₂)ₙ—O—CH₃, with n=3, 4, 5, 6,
Z is 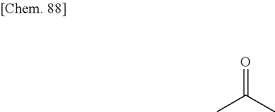
or —CH₂—,
Y═—OH; —OMe;-OEt; —OPr; —NH₂; —NHMe; —N(Me)₂; —N(Me)OCH₃; —NH—(CH₂)₂-(3-indolyl); —NH(CH₂)₂-3-((5-hydroxy)indolyl); —NH—CH(R₃)—COR₂, with R₂ selected from:
OH; —OMe;-OEt; —OPr; —NH₂; —NHMe; —N(Me)₂; —N(Me)OCH₃; 3-(5-methoxy)indolyl; —NH—(CH₂)₂-(3-indolyl); —NH(CH₂)₂-3-((5-hydroxy)indolyl); —NH(CH₂)₂-3-((5-methoxy)indolyl) 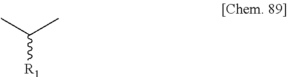
in formula (I) and R₃ of the substituent —NH—CH(R₃)—COR₂ of Y are independently selected from: H or

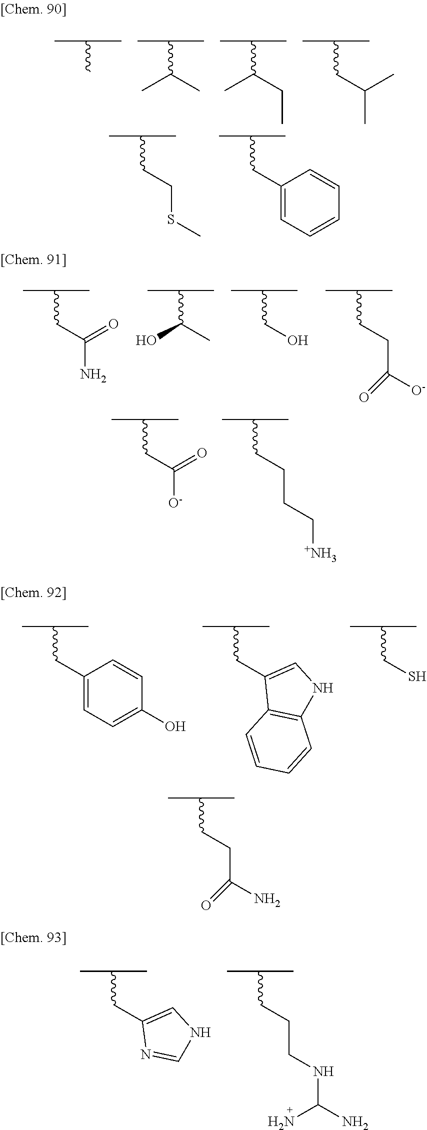

7. The method according to claim 6, wherein the ring A is substituted in the 2-, 3-, 4-, 5-position by one or two Br and Y═—OH; —OMe; —NH—(CH₂)₂-(3-indolyl); —NH(CH₂)₂-3-((5-hydroxy)indolyl); —NH—CH(R₃)—COR₂, R₁, R₂ and R₃ being as defined in claim 6.

8. The method according to claim 6, wherein Y is —NH—(CH₂)₂-(3-indolyl) or —NH(CH₂)₂-3-((5-hydroxy)indolyl) with the proviso that:

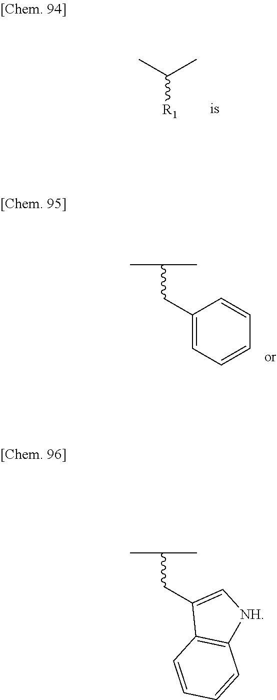

9. The method according to claim 6, selected from:
methyl (5-((2-bromobenzyl)oxy)-4-oxo-4H-chromene-2-carbonylamino)-L-alloisoleucinate;
methyl (5-((4-bromobenzyl)oxy)-4-oxo-4H-chromene-2-carbonylamino)-L-alloisoleucinate;
methyl (5-((2-bromobenzyl)oxy)-4-oxo-4H-chromene-2-carbonylamino)-L-leucinate;
methyl (5-((2-bromobenzyl)oxy)-4-oxo-4H-chromene-2-carbonylamino)-L-valinate;
methyl (5-((2-bromobenzyl)oxy)-4-oxo-4H-chromene-2-carbonylamino)-L-phenylalaninate;
methyl (5-((4-bromobenzyl)oxy)-4-oxo-4H-chromene-2-carbonylamino)-L-phenylalaninate;
methyl (5-((2-bromobenzyl)oxy)-4-oxo-4H-chromene-2-carbonylamino)-L-tryptophanate;
methyl (5-((4-bromobenzyl)oxy)-4-oxo-4H-chromene-2-carbonylamino)-L-tryptophanate;
methyl (5-((4-bromobenzyl)oxy)-4-oxo-4H-chromene-2-carbonylamino)-D-tryptophanate;
(5-((2-bromobenzyl)oxy)-4-oxo-4H-chromene-2-carbonylamino)-L-alloisoleucine;
(5-((2-bromobenzyl)oxy)-4-oxo-4H-chromene-2-carbonylamino)-L-phenylalanine;
(5-((4-bromobenzyl)oxy)-4-oxo-4H-chromene-2-carbonylamino)-L-phenylalanine;
(5-((4-bromobenzyl)oxy)-4-oxo-4H-chromene-2-carbonylamino)-D-tryptophan;
methyl (5-((2-bromobenzyl)oxy)-4-oxo-4H-chromene-2-carbonylamino)-L-alloisoleucyl-L-valinate;
methyl (5-((2-bromobenzyl)oxy)-4-oxo-4H-chromene-2-carbonylamino)-L-alloisoleucyl-L-leucinate;
methyl (5-((4-bromobenzyl)oxy)-4-oxo-4H-chromene-2-carbonylamino)-L-alloisoleucyl-L-valinate;
methyl (5-((4-bromobenzyl)oxy)-4-oxo-4H-chromene-2-carbonylamino)-L-alloisoleucyl-L-leucinate;
(S)-5-((2-bromobenzyl)oxy)-N-(1-((2-(5-hydroxy-1H-indol-3-yl)ethyl)amino)-1-oxo-3-phenylpropan-2-yl)-4-oxo-4H-chromene-2-carboxamide;
(S)—N-(1-((2-(1H-indol-3-yl)ethyl)amino)-1-oxo-3-phenylpropan-2-yl)-5-((4-bromobenzyl)oxy)-4-oxo-4H-chromene-2-carboxamide, and
(R)—N-(1-((2-(1H-indol-3-yl)ethyl)amino)-3-(1H-indol-3-yl)-1-oxopropan-2-yl)-5-((4-bromobenzyl)oxy)-4-oxo-4H-chromene-2-carboxamide.

10. A pharmaceutical composition comprising:
at least one pharmaceutically acceptable active agent; and
at least one compound according to one claim 1.

11. The pharmaceutical composition of claim 10, wherein the pharmaceutically acceptable active agent is selected from anti-cancer agents, intestinal anti-inflammatory agents, hypocholesteremic agents, anti-dyslipidemic agents and kinase inhibitors.
